SEQUENCE LISTING (337 sequences)

SEQ ID NO: 1 - DNA, 20 bp, Articifial Sequence
  Synthetic Oligonucleotide
ctggagcagctgcctctagg

SEQ ID NO: 2 - DNA, 20 bp, Artificial Sequence
  Antisense Oligonucleotide
ccctgcatgaagctgagaag

SEQ ID NO: 3 - DNA, 20 bp, Artificial Sequence
  Antisense Oligonucleotide
gtgcttcatgtaaccctgca

SEQ ID NO: 4 - DNA, 20 bp, Artificial Sequence
  Antisense Oligonucleotide
tggcctgctgggccacctgg

SEQ ID NO: 5 - DNA, 20 bp, Artificial Sequence
  Antisense Oligonucleotide
tgctccagtagtctttcagg

SEQ ID NO: 6 - DNA, 20 bp, Artificial Sequence
  Antisense Oligonucleotide
tgacctcagggtccaaatcc

SEQ ID NO: 7 - DNA, 20 bp, Artificial Sequence
  Antisense Oligonucleotide
ctctagggatgaactgagca

SEQ ID NO: 8 - DNA, 20 bp, Artificial Sequence
  Antisense Oligonucleotide
cagctgcctctagggatgaa

SEQ ID NO: 9 - DNA, 20 bp, Artificial Sequence
  Antisense Oligonucleotide
ttcctggagcagctgcctct

SEQ ID NO: 10 - DNA, 20 bp, Artificial Sequence
  Antisense Oligonucleotide
acctctgttcctggagcagc

SEQ ID NO: 11 - DNA, 20 bp, Artificial Sequence
  Antisense Oligonucleotide
atggcacctctgttcctgga

SEQ ID NO: 12 - DNA, 20 bp, Artificial Sequence
  Antisense Oligonucleotide
gggctgcatggcacctctgt

SEQ ID NO: 13 - DNA, 20 bp, Artificial Sequence
  Antisense Oligonucleotide
ggcaacaacaaggagtaccc

SEQ ID NO: 14 - DNA, 20 bp, Artificial Sequence
  Antisense Oligonucleotide
ggagggcaacaacaaggagt

SEQ ID NO: 15 - DNA, 20 bp, Artificial Sequence
  Antisense Oligonucleotide
agctcgggcagaggccagga

SEQ ID NO: 16 - DNA, 20 bp, Artificial Sequence
  Antisense Oligonucleotide
tctgaagctcgggcagaggc

SEQ ID NO: 17 - DNA, 20 bp, Artificial Sequence
  Antisense Oligonucleotide
cggcctctgaagctcgggca

SEQ ID NO: 18 - DNA, 20 bp, Artificial Sequence
  Antisense Oligonucleotide
catcctcggcctctgaagct

SEQ ID NO: 19 - DNA, 20 bp, Artificial Sequence
  Antisense Oligonucleotide
gggaggcatcctcggcctct

SEQ ID NO: 20 - DNA, 20 bp, Artificial Sequence
  Antisense Oligonucleotide
gagaagggaggcatcctcgg

SEQ ID NO: 21 - DNA, 20 bp, Artificial Sequence
  Antisense Oligonucleotide
gctgagaagggaggcatcct

SEQ ID NO: 22 - DNA, 20 bp, Artificial Sequence
  Antisense Oligonucleotide
tgcatgaagctgagaaggga

SEQ ID NO: 23 - DNA, 20 bp, Artificial Sequence
  Antisense Oligonucleotide
gcgtgcttcatgtaaccctg

SEQ ID NO: 24 - DNA, 20 bp, Artificial Sequence
  Antisense Oligonucleotide
ttggtggcgtgcttcatgta

SEQ ID NO: 25 - DNA, 20 bp, Artificial Sequence
  Antisense Oligonucleotide
gcatccttggcggtcttggt

SEQ ID NO: 26 - DNA, 20 bp, Artificial Sequence
  Antisense Oligonucleotide
ctcagtgcatccttggcggt

SEQ ID NO: 27 - DNA, 20 bp, Artificial Sequence
  Antisense Oligonucleotide
tgctcagtgcatccttggcy

SEQ ID NO: 28 - DNA, 20 bp, Artificial Sequence
  Antisense Oligonucleotide
ctcctgcacgctgctcagtg

SEQ ID NO: 29 - DNA, 20 bp, Artificial Sequence
  Antisense Oligonucleotide
gactcctgcacgctgctcag

SEQ ID NO: 30 - DNA, 20 bp, Artificial Sequence
  Antisense Oligonucleotide
gccacctgggactcctgcac

SEQ ID NO: 31 - DNA, 20 bp, Artificial Sequence
  Antisense Oligonucleotide
gcccctggcctgctgggcca

SEQ ID NO: 32 - DNA, 20 bp, Artificial Sequence
  Antisense Oligonucleotide
agcccctggcctgctgggcc

SEQ ID NO: 33 - DNA, 20 bp, Artificial Sequence
  Antisense Oligonucleotide
gaagccatcggtcacccagc

SEQ ID NO: 34 - DNA, 20 bp, Artificial Sequence
  Antisense Oligonucleotide
ctgaagccatcggtcaccca

SEQ ID NO: 35 - DNA, 20 bp, Artificial Sequence
  Antisense Oligonucleotide
tttcagggaactgaagccat

SEQ ID NO: 36 - DNA, 20 bp, Artificial Sequence
  Antisense Oligonucleotide
cagtagtctttcagggaact

SEQ ID NO: 37 - DNA, 20 bp, Artificial Sequence
  Antisense Oligonucleotide
aacggtgctccagtagtctt

SEQ ID NO: 38 - DNA, 20 bp, Artificial Sequence
  Antisense Oligonucleotide
ccttaacggtgctccagtag

SEQ ID NO: 39 - DNA, 20 bp, Artificial Sequence
  Antisense Oligonucleotide
gaacttgtccttaacggtgc

SEQ ID NO: 40 - DNA, 20 bp, Artificial Sequence
  Antisense Oligonucleotide
ctcagagaacttgtccttaa

SEQ ID NO: 41 - DNA, 20 bp, Artificial Sequence
  Antisense Oligonucleotide
agaactcagagaacttgtcc

SEQ ID NO: 42 - DNA, 20 bp, Artificial Sequence
  Antisense Oligonucleotide
atcccagaactcagagaact

SEQ ID NO: 43 - DNA, 20 bp, Artificial Sequence
  Antisense Oligonucleotide
cagggtccaaatcccagaac

SEQ ID NO: 44 - DNA, 20 bp, Artificial Sequence
  Antisense Oligonucleotide
ttggtctgacctcagggtcc

SEQ ID NO: 45 - DNA, 20 bp, Artificial Sequence
  Antisense Oligonucleotide
gttggtctgacctcagggtc

SEQ ID NO: 46 - DNA, 20 bp, Artificial Sequence
  Antisense Oligonucleotide
gctgaagttggtctgacctc

SEQ ID NO: 47 - DNA, 20 bp, Artificial Sequence
  Antisense Oligonucleotide
cagccacggctgaagttggt

SEQ ID NO: 48 - DNA, 20 bp, Artificial Sequence
  Antisense Oligonucleotide
caggcagccacggctgaagt

SEQ ID NO: 49 - DNA, 20 bp, Artificial Sequence
  Antisense Oligonucleotide
attgaggtctcaggcagcca

SEQ ID NO: 50 - DNA, 20 bp, Artificial Sequence
  Antisense Oligonucleotide
tggataggcaggtggacttg

SEQ ID NO: 51 - DNA, 20 bp, Artificial Sequence
  Antisense Oligonucleotide
ctcgcaggatggataggcag

SEQ ID NO: 52 - DNA, 20 bp, Artificial Sequence
  Antisense Oligonucleotide
aggagctcgcaggatggata

SEQ ID NO: 53 - DNA, 20 bp, Artificial Sequence
  Antisense Oligonucleotide
gacccaaggagctcgcagga

SEQ ID NO: 54 - DNA, 20 bp, Artificial Sequence
  Antisense Oligonucleotide
tgcaggacccaaggagctcg

SEQ ID NO: 55 - DNA, 20 bp, Artificial Sequence
  Antisense Oligonucleotide
tggagattgcaggacccaag

SEQ ID NO: 56 - DNA, 20 bp, Artificial Sequence
  Antisense Oligonucleotide
agccctggagattgcaggac

SEQ ID NO: 57 - DNA, 20 bp, Artificial Sequence
  Antisense Oligonucleotide
ggcagccctggagattgcag

SEQ ID NO: 58 - DNA, 20 bp, Artificial Sequence
  Antisense Oligonucleotide
ccttttaagcaacctacagg

SEQ ID NO: 59 - DNA, 20 bp, Artificial Sequence
  Antisense Oligonucleotide
ctgtcccttttaagcaacct

SEQ ID NO: 60 - DNA, 20 bp, Artificial Sequence
  Antisense Oligonucleotide
agaatactgtcccttttaag

SEQ ID NO: 61 - DNA, 20 bp, Artificial Sequence
  Antisense Oligonucleotide
cactgagaatactgtccctt

SEQ ID NO: 62 - DNA, 20 bp, Artificial Sequence
  Antisense Oligonucleotide
taggagagcactgagaatac

SEQ ID NO: 63 - DNA, 20 bp, Artificial Sequence
  Antisense Oligonucleotide
gggtaggagagcactgagaa

SEQ ID NO: 64 - DNA, 20 bp, Artificial Sequence
  Antisense Oligonucleotide
aggccagcatgcctggaggg

SEQ ID NO: 65 - DNA, 20 bp, Artificial Sequence
  Antisense Oligonucleotide
ttgggaggccagcatgcctg

SEQ ID NO: 66 - DNA, 20 bp, Artificial Sequence
  Antisense Oligonucleotide
agctttattgggaggccagc

SEQ ID NO: 67 - DNA, 20 bp, Artificial Sequence
  Antisense Oligonucleotide
tgtccagctttattgggagg

SEQ ID NO: 68 - DNA, 20 bp, Artificial Sequence
  Antisense Oligonucleotide
cttgtccagctttattggga

SEQ ID NO: 69 - DNA, 20 bp, Artificial Sequence
  Antisense Oligonucleotide
agcttcttgtccagctttat

SEQ ID NO: 70 - DNA, 20 bp, Artificial Sequence
  Antisense Oligonucleotide
catagcagcttcttgtccag

SEQ ID NO: 71 - DNA, 20 bp, Artificial Sequence
  Antisense Oligonucleotide
acctggagcagctgcctcta

SEQ ID NO: 72 - DNA, 20 bp, Artificial Sequence
  Antisense Oligonucleotide
agggcattacctggagcagc

SEQ ID NO: 73 - DNA, 20 bp, Artificial Sequence
  Antisense Oligonucleotide
acctctgttcctgcaaggaa

SEQ ID NO: 74 - DNA, 20 bp, Artificial Sequence
  Antisense Oligonucleotide
aagtgcttacgggcagaggc

SEQ ID NO: 75 - DNA, 20 bp, Artificial Sequence
  Antisense Oligonucleotide
gcgggtgtacctggcctgct

SEQ ID NO: 76 - DNA, 19 bp, Artificial Sequence
  Antisense Oligonucleotide
aaccctttgtgaactgcac

SEQ ID NO: 77 - DNA, 20 bp, Artificial Sequence
  Antisense Oligonucleotide
agtgagcaataccgcctgag

SEQ ID NO: 78 - DNA, 19 bp, Artificial Sequence
  Antisense Oligonucleotide
cggcttgaattaggtcagg

SEQ ID NO: 79 - DNA, 20 bp, Artificial Sequence
  Antisense Oligonucleotide
tagggataaaactgagcagg

SEQ ID NO: 80 - DNA, 20 bp, Artificial Sequence
  Antisense Oligonucleotide
ctggagtagctagctgcttc

SEQ ID NO: 81 - DNA, 20 bp, Artificial Sequence
  Antisense Oligonucleotide
gctgcatggcacctacgtac

SEQ ID NO: 82 - DNA, 20 bp, Artificial Sequence
  Antisense Oligonucleotide
ccacagtgaggagcgtccgg

SEQ ID NO: 83 - DNA, 20 bp, Artificial Sequence
  Antisense Oligonucleotide
ggcagatgccaggagagcca

SEQ ID NO: 84 - DNA, 20 bp, Artificial Sequence
  Antisense Oligonucleotide
ctacctcttcagctcgggca

SEQ ID NO: 85 - DNA, 20 bp, Artificial Sequence
  Antisense Oligonucleotide
cagcagcaaggatccctcta

SEQ ID NO: 86 - DNA, 20 bp, Artificial Sequence
  Antisense Oligonucleotide
gcacagagcccagcagcaag

SEQ ID NO: 87 - DNA, 20 bp, Artificial Sequence
  Antisense Oligonucleotide
ccctggccaccgcagctata

SEQ ID NO: 88 - DNA, 20 bp, Artificial Sequence
  Antisense Oligonucleotide
atctgaagtgattgtccatc

SEQ ID NO: 89 - DNA, 20 bp, Artificial Sequence
  Antisense Oligonucleotide
agtagcctttcaggaatctg

SEQ ID NO: 90 - DNA, 20 bp, Artificial Sequence
  Antisense Oligonucleotide
cttgtcagtaaacttgctcc

SEQ ID NO: 91 - DNA, 20 bp, Artificial Sequence
  Antisense Oligonucleotide
gaagccggtgaacttgtcag

SEQ ID NO: 92 - DNA, 20 bp, Artificial Sequence
  Antisense Oligonucleotide
gaatcccagaagccggtgaa

SEQ ID NO: 93 - DNA, 20 bp, Artificial Sequence
  Antisense Oligonucleotide
ggttagaatcccagaagccg

SEQ ID NO: 94 - DNA, 20 bp, Artificial Sequence
  Antisense Oligonucleotide
tggagttggttggtcctcag

SEQ ID NO: 95 - DNA, 20 bp, Artificial Sequence
  Antisense Oligonucleotide
tcacgactcaatagctggag

SEQ ID NO: 96 - DNA, 20 bp, Artificial Sequence
  Antisense Oligonucleotide
cccttaaagcaaccttcagg

SEQ ID NO: 97 - DNA, 20 bp, Artificial Sequence
  Antisense Oligonucleotide
agacatgagaacatactttc

SEQ ID NO: 98 - DNA, 20 bp, Artificial Sequence
  Antisense Oligonucleotide
catgtttaggtgagatctag

SEQ ID NO: 99 - DNA, 20 bp, Artificial Sequence
  Antisense Oligonucleotide
tcttatccagctttattagg

SEQ ID NO: 100 - DNA, 20 bp, Artificial Sequence
  Antisense Oligonucleotide
gctgcatggcacctctgttc

SEQ ID NO: 101 - DNA, 20 bp, Artificial Sequence
  Antisense Oligonucleotide
ggcagaggccaggagcgcca

SEQ ID NO: 102 - DNA, 20 bp, Artificial Sequence
  Antisense Oligonucleotide
ctgaagctcgggcagaggcc

SEQ ID NO: 103 - DNA, 20 bp, Artificial Sequence
  Antisense Oligonucleotide
tcctcggcctctgaagctcg

SEQ ID NO: 104 - DNA, 20 bp, Artificial Sequence
  Antisense Oligonucleotide
tcttggtggcgtgcttcatg

SEQ ID NO: 105 - DNA, 20 bp, Artificial Sequence
  Antisense Oligonucleotide
gctcagtgcatccttggcgg

SEQ ID NO: 106 - DNA, 20 bp, Artificial Sequence
  Antisense Oligonucleotide
cctgcacgctgctcagtgca

SEQ ID NO: 107 - DNA, 20 bp, Artificial Sequence
  Antisense Oligonucleotide
actgaagccatcggtcaccc

SEQ ID NO: 108 - DNA, 20 bp, Artificial Sequence
  Antisense Oligonucleotide
cagaactcagagaacttgtc

SEQ ID NO: 109 - DNA, 20 bp, Artificial Sequence
  Antisense Oligonucleotide
gaagttggtctgacctcagg

SEQ ID NO: 110 - DNA, 20 bp, Artificial Sequence
  Antisense Oligonucleotide
ccctggagattgcaggaccc

SEQ ID NO: 111 - DNA, 20 bp, Artificial Sequence
  Antisense Oligonucleotide
gggcagccctggagattgca

SEQ ID NO: 112 - DNA, 20 bp, Artificial Sequence
  Antisense Oligonucleotide
cccttttaagcaacctacag

SEQ ID NO: 113 - DNA, 20 bp, Artificial Sequence
  Antisense Oligonucleotide
tgaacttatcagtgaacttg

SEQ ID NO: 114 - DNA, 20 bp, Artificial Sequence
  Antisense Oligonucleotide
tcagggccagactcccagag

SEQ ID NO: 115 - DNA, 20 bp, Artificial Sequence
  Antisense Oligonucleotide
ttggtgttgttagttggtcc

SEQ ID NO: 116 - DNA, 20 bp, Artificial Sequence
  Antisense Oligonucleotide
ttggtgttgttagttggtcc

SEQ ID NO: 117 - DNA, 20 bp, Artificial Sequence
  Antisense Oligonucleotide
agagccacgagggccacgat

SEQ ID NO: 118 - DNA, 20 bp, Artificial Sequence
  Antisense Oligonucleotide
agaggccaggagagccacga

SEQ ID NO: 119 - DNA, 20 bp, Artificial Sequence
  Antisense Oligonucleotide
cagctcgggcagaggccagg

SEQ ID NO: 120 - DNA, 20 bp, Artificial Sequence
  Antisense Oligonucleotide
tctccctcatcagctcgggc

SEQ ID NO: 121 - DNA, 20 bp, Artificial Sequence
  Antisense Oligonucleotide
gcccagcagcaaggatccct

SEQ ID NO: 122 - DNA, 20 bp, Artificial Sequence
  Antisense Oligonucleotide
cctgcatagagcccagcagc

SEQ ID NO: 123 - DNA, 20 bp, Artificial Sequence
  Antisense Oligonucleotide
tccatgtagccctgcataga

SEQ ID NO: 124 - DNA, 20 bp, Artificial Sequence
  Antisense Oligonucleotide
ggaccgtcttggaggcttgt

SEQ ID NO: 125 - DNA, 20 bp, Artificial Sequence
  Antisense Oligonucleotide
agtgcatcctggaccgtctt

SEQ ID NO: 126 - DNA, 20 bp, Artificial Sequence
  Antisense Oligonucleotide
catgctgcttagtgcatcct

SEQ ID NO: 127 - DNA, 20 bp, Artificial Sequence
  Antisense Oligonucleotide
cagactcctgcatgctgctt

SEQ ID NO: 128 - DNA, 20 bp, Artificial Sequence
  Antisense Oligonucleotide
acagctatatcagactcctg

SEQ ID NO: 129 - DNA, 20 bp, Artificial Sequence
  Antisense Oligonucleotide
ctggccaccacagctatatc

SEQ ID NO: 130 - DNA, 20 bp, Artificial Sequence
  Antisense Oligonucleotide
aagcgattgtccatccagcc

SEQ ID NO: 131 - DNA, 20 bp, Artificial Sequence
  Antisense Oligonucleotide
tgctccagtagcctttcagg

SEQ ID NO: 132 - DNA, 20 bp, Artificial Sequence
  Antisense Oligonucleotide
gaacttgctccagtagcctt

SEQ ID NO: 133 - DNA, 20 bp, Artificial Sequence
  Antisense Oligonucleotide
cagtgaacttgctccagtag

SEQ ID NO: 134 - DNA, 20 bp, Artificial Sequence
  Antisense Oligonucleotide
cttatcagtgaacttgctcc

SEQ ID NO: 135 - DNA, 20 bp, Artificial Sequence
  Antisense Oligonucleotide
ccagtgaacttatcagtgaa

SEQ ID NO: 136 - DNA, 20 bp, Artificial Sequence
  Antisense Oligonucleotide
gaggccagtgaacttatcag

SEQ ID NO: 137 - DNA, 20 bp, Artificial Sequence
  Antisense Oligonucleotide
ccagaggccagtgaacttat

SEQ ID NO: 138 - DNA, 20 bp, Artificial Sequence
  Antisense Oligonucleotide
gactcccagaggccagtgaa

SEQ ID NO: 139 - DNA, 20 bp, Artificial Sequence
  Antisense Oligonucleotide
ggccagactcccagaggcca

SEQ ID NO: 140 - DNA, 20 bp, Artificial Sequence
  Antisense Oligonucleotide
agttggtcctcagggccaga

SEQ ID NO: 141 - DNA, 20 bp, Artificial Sequence
  Antisense Oligonucleotide
gttagttggtcctcagggcc

SEQ ID NO: 142 - DNA, 20 bp, Artificial Sequence
  Antisense Oligonucleotide
tgttgttagttggtcctcag

SEQ ID NO: 143 - DNA, 20 bp, Artificial Sequence
  Antisense Oligonucleotide
agagttggtgttgttagttg

SEQ ID NO: 144 - DNA, 20 bp, Artificial Sequence
  Antisense Oligonucleotide
gctcaagagttggtgttgtt

SEQ ID NO: 145 - DNA, 20 bp, Artificial Sequence
  Antisense Oligonucleotide
cacggctcaagagttggtgt

SEQ ID NO: 146 - DNA, 20 bp, Artificial Sequence
  Antisense Oligonucleotide
gtctcacggctcaagagttg

SEQ ID NO: 147 - DNA, 20 bp, Artificial Sequence
  Antisense Oligonucleotide
gaacatggaggtctcacggc

SEQ ID NO: 148 - DNA, 20 bp, Artificial Sequence
  Antisense Oligonucleotide
tctggaacatggaggtctca

SEQ ID NO: 149 - DNA, 20 bp, Artificial Sequence
  Antisense Oligonucleotide
cacatctggaacatggaggt

SEQ ID NO: 150 - DNA, 20 bp, Artificial Sequence
  Antisense Oligonucleotide
cagacacatctggaacatgg

SEQ ID NO: 151 - DNA, 20 bp, Artificial Sequence
  Antisense Oligonucleotide
tggccagacacatctggaac

SEQ ID NO: 152 - DNA, 20 bp, Artificial Sequence
  Antisense Oligonucleotide
aggatagatggccagacaca

SEQ ID NO: 153 - DNA, 20 bp, Artificial Sequence
  Antisense Oligonucleotide
cagcaggatagatggccaga

SEQ ID NO: 154 - DNA, 20 bp, Artificial Sequence
  Antisense Oligonucleotide
gaggcagcaggatagatggc

SEQ ID NO: 155 - DNA, 20 bp, Artificial Sequence
  Antisense Oligonucleotide
ttcggaggcagcaggataga

SEQ ID NO: 156 - DNA, 20 bp, Artificial Sequence
  Antisense Oligonucleotide
aaccttcggaggcagcagga

SEQ ID NO: 157 - DNA, 20 bp, Artificial Sequence
  Antisense Oligonucleotide
gagcaaccttcggaggcagc

SEQ ID NO: 158 - DNA, 20 bp, Artificial Sequence
  Antisense Oligonucleotide
cttagagcaaccttcggagg

SEQ ID NO: 159 - DNA, 20 bp, Artificial Sequence
  Antisense Oligonucleotide
tccccttagagcaaccttcg

SEQ ID NO: 160 - DNA, 20 bp, Artificial Sequence
  Antisense Oligonucleotide
actttccccttagagcaacc

SEQ ID NO: 161 - DNA, 20 bp, Artificial Sequence
  Antisense Oligonucleotide
atatactttccccttagagc

SEQ ID NO: 162 - DNA, 20 bp, Artificial Sequence
  Antisense Oligonucleotide
gagaatatactttcccctta

SEQ ID NO: 163 - DNA, 20 bp, Artificial Sequence
  Antisense Oligonucleotide
gcatgagaatatactttccc

SEQ ID NO: 164 - DNA, 20 bp, Artificial Sequence
  Antisense Oligonucleotide
aaaggcatgagaatatactt

SEQ ID NO: 165 - DNA, 20 bp, Artificial Sequence
  Antisense Oligonucleotide
ggataaaggcatgagaatat

SEQ ID NO: 166 - DNA, 20 bp, Artificial Sequence
  Antisense Oligonucleotide
ggagggataaaggcatgaga

SEQ ID NO: 167 - DNA, 20 bp, Artificial Sequence
  Antisense Oligonucleotide
gcatgtttaggtgaggtctg

SEQ ID NO: 168 - DNA, 20 bp, Artificial Sequence
  Antisense Oligonucleotide
gacagcatgtttaggtgagg

SEQ ID NO: 169 - DNA, 20 bp, Artificial Sequence
  Antisense Oligonucleotide
ttatttgggacagcatgttt

SEQ ID NO: 170 - DNA, 20 bp, Artificial Sequence
  Antisense Oligonucleotide
gcttttatttgggacagcat

SEQ ID NO: 171 - DNA, 20 bp, Artificial Sequence
  Antisense Oligonucleotide
tcccagcttttatttgggac

SEQ ID NO: 172 - DNA, 20 bp, Artificial Sequence
  Antisense Oligonucleotide
cacgatgaggagcattcggg

SEQ ID NO: 173 - DNA, 20 bp, Artificial Sequence
  Antisense Oligonucleotide
agggccacgatgaggagcat

SEQ ID NO: 174 - DNA, 20 bp, Artificial Sequence
  Antisense Oligonucleotide
ccacgagggccacgatgagg

SEQ ID NO: 175 - DNA, 20 bp, Artificial Sequence
  Antisense Oligonucleotide
gagagccacgagggccacga

SEQ ID NO: 176 - DNA, 20 bp, Artificial Sequence
  Antisense Oligonucleotide
gccaggagagccacgagggc

SEQ ID NO: 177 - DNA, 20 bp, Artificial Sequence
  Antisense Oligonucleotide
cagaggccaggagagccacg

SEQ ID NO: 178 - DNA, 20 bp, Artificial Sequence
  Antisense Oligonucleotide
tcgggcagaggccaggagag

SEQ ID NO: 179 - DNA, 20 bp, Artificial Sequence
  Antisense Oligonucleotide
tcagctcgggcagaggccag

SEQ ID NO: 180 - DNA, 20 bp, Artificial Sequence
  Antisense Oligonucleotide
cctcatcagctcgggcagag

SEQ ID NO: 181 - DNA, 20 bp, Artificial Sequence
  Antisense Oligonucleotide
ctctccctcatcagctcggg

SEQ ID NO: 182 - DNA, 20 bp, Artificial Sequence
  Antisense Oligonucleotide
gatccctctccctcatcagc

SEQ ID NO: 183 - DNA, 20 bp, Artificial Sequence
  Antisense Oligonucleotide
gcaaggatccctctccctca

SEQ ID NO: 184 - DNA, 20 bp, Artificial Sequence
  Antisense Oligonucleotide
cagcagcaaggatccctctc

SEQ ID NO: 185 - DNA, 20 bp, Artificial Sequence
  Antisense Oligonucleotide
gagcccagcagcaaggatcc

SEQ ID NO: 186 - DNA, 20 bp, Artificial Sequence
  Antisense Oligonucleotide
gcatagagcccagcagcaag

SEQ ID NO: 187 - DNA, 20 bp, Artificial Sequence
  Antisense Oligonucleotide
gccctgcatagagcccagca

SEQ ID NO: 188 - DNA, 20 bp, Artificial Sequence
  Antisense Oligonucleotide
atgtagccctgcatagagcc

SEQ ID NO: 189 - DNA, 20 bp, Artificial Sequence
  Antisense Oligonucleotide
gttccatgtagccctgcata

SEQ ID NO: 190 - DNA, 20 bp, Artificial Sequence
  Antisense Oligonucleotide
ggcttgttccatgtagccct

SEQ ID NO: 191 - DNA, 20 bp, Artificial Sequence
  Antisense Oligonucleotide
ttggaggcttgttccatgta

SEQ ID NO: 192 - DNA, 20 bp, Artificial Sequence
  Antisense Oligonucleotide
ccgtcttggaggcttgttcc

SEQ ID NO: 193 - DNA, 20 bp, Artificial Sequence
  Antisense Oligonucleotide
ctggaccgtcttggaggctt

SEQ ID NO: 194 - DNA, 20 bp, Artificial Sequence
  Antisense Oligonucleotide
gcatcctggaccgtcttgga

SEQ ID NO: 195 - DNA, 20 bp, Artificial Sequence
  Antisense Oligonucleotide
ttagtgcatcctggaccgtc

SEQ ID NO: 196 - DNA, 20 bp, Artificial Sequence
  Antisense Oligonucleotide
gctgcttagtgcatcctgga

SEQ ID NO: 197 - DNA, 20 bp, Artificial Sequence
  Antisense Oligonucleotide
tgcatgctgcttagtgcatc

SEQ ID NO: 198 - DNA, 20 bp, Artificial Sequence
  Antisense Oligonucleotide
actcctgcatgctgcttagt

SEQ ID NO: 199 - DNA, 20 bp, Artificial Sequence
  Antisense Oligonucleotide
atcagactcctgcatgctgc

SEQ ID NO: 200 - DNA, 20 bp, Artificial Sequence
  Antisense Oligonucleotide
gctatatcagactcctgcat

SEQ ID NO: 201 - DNA, 20 bp, Artificial Sequence
  Antisense Oligonucleotide
ccacagctatatcagactcc

SEQ ID NO: 202 - DNA, 20 bp, Artificial Sequence
  Antisense Oligonucleotide
ggccaccacagctatatcag

SEQ ID NO: 203 - DNA, 20 bp, Artificial Sequence
  Antisense Oligonucleotide
ctgctggccaccacagctat

SEQ ID NO: 204 - DNA, 20 bp, Artificial Sequence
  Antisense Oligonucleotide
agcccctgctggccaccaca

SEQ ID NO: 205 - DNA, 20 bp, Artificial Sequence
  Antisense Oligonucleotide
catccagcccctgctggcca

SEQ ID NO: 206 - DNA, 20 bp, Artificial Sequence
  Antisense Oligonucleotide
ttgtccatccagcccctgct

SEQ ID NO: 207 - DNA, 20 bp, Artificial Sequence
  Antisense Oligonucleotide
attgtccatccagcccctgc

SEQ ID NO: 208 - DNA, 20 bp, Artificial Sequence
  Antisense Oligonucleotide
agcgattgtccatccagccc

SEQ ID NO: 209 - DNA, 20 bp, Artificial Sequence
  Antisense Oligonucleotide
tttgaagcgattgtccatcc

SEQ ID NO: 210 - DNA, 20 bp, Artificial Sequence
  Antisense Oligonucleotide
agggatttgaagcgattgtc

SEQ ID NO: 211 - DNA, 20 bp, Artificial Sequence
  Antisense Oligonucleotide
ctttcagggatttgaagcga

SEQ ID NO: 212 - DNA, 20 bp, Artificial Sequence
  Antisense Oligonucleotide
gtagcctttcagggatttga

SEQ ID NO: 213 - DNA, 20 bp, Artificial Sequence
  Antisense Oligonucleotide
ctccagtagcctttcaggga

SEQ ID NO: 214 - DNA, 20 bp, Artificial Sequence
  Antisense Oligonucleotide
acttgctccagtagcctttc

SEQ ID NO: 215 - DNA, 20 bp, Artificial Sequence
  Antisense Oligonucleotide
agtgaacttgctccagtagc

SEQ ID NO: 216 - DNA, 20 bp, Artificial Sequence
  Antisense Oligonucleotide
ttatcagtgaacttgctcca

SEQ ID NO: 217 - DNA, 20 bp, Artificial Sequence
  Antisense Oligonucleotide
gccagtcaacttatcagtga

SEQ ID NO: 218 - DNA, 20 bp, Artificial Sequence
  Antisense Oligonucleotide
cagaggccagtgaacttatc

SEQ ID NO: 219 - DNA, 20 bp, Artificial Sequence
  Antisense Oligonucleotide
actcccagaggccagtgaac

SEQ ID NO: 220 - DNA, 20 bp, Artificial Sequence
  Antisense Oligonucleotide
gccagactcccagaggccag

SEQ ID NO: 221 - DNA, 20 bp, Artificial Sequence
  Antisense Oligonucleotide
tagttggtcctcagggccag

SEQ ID NO: 222 - DNA, 20 bp, Artificial Sequence
  Antisense Oligonucleotide
gttgttagttggtcctcagg

SEQ ID NO: 223 - DNA, 20 bp, Artificial Sequence
  Antisense Oligonucleotide
aagagttggtgttgttagtt

SEQ ID NO: 224 - DNA, 20 bp, Artificial Sequence
  Antisense Oligonucleotide
ggctcaagagttggtgttgt

SEQ ID NO: 225 - DNA, 20 bp, Artificial Sequence
  Antisense Oligonucleotide
tcacggctcaagagttggtg

SEQ ID NO: 226 - DNA, 20 bp, Artificial Sequence
  Antisense Oligonucleotide
ggaggcttgttccatgtagc

SEQ ID NO: 227 - DNA, 20 bp, Artificial Sequence
  Antisense Oligonucleotide
tcagggatttgaagcgattg

SEQ ID NO: 228 - DNA, 20 bp, Artificial Sequence
  Antisense Oligonucleotide
cagtagcctttcagggattt

SEQ ID NO: 229 - DNA, 20 bp, Artificial Sequence
  Antisense Oligonucleotide
atggaggtctcacggctcaa

SEQ ID NO: 230 - DNA, 20 bp, Artificial Sequence
  Antisense Oligonucleotide
tagatggccagacacatctg

SEQ ID NO: 231 - DNA, 20 bp, Artificial Sequence
  Antisense Oligonucleotide
ttgggacagcatgtttaggt

SEQ ID NO: 232 - DNA, 20 bp, Artificial Sequence
  Antisense Oligonucleotide
tgccaagagggcaacaatag

SEQ ID NO: 233 - DNA, 20 bp, Artificial Sequence
  Antisense Oligonucleotide
aggagtgccaagagggcaac

SEQ ID NO: 234 - DNA, 20 bp, Artificial Sequence
  Antisense Oligonucleotide
gatgccaggagtgccaagag

SEQ ID NO: 235 - DNA, 20 bp, Artificial Sequence
  Antisense Oligonucleotide
ggcagatgccaggagtgcca

SEQ ID NO: 236 - DNA, 20 bp, Artificial Sequence
  Antisense Oligonucleotide
ctctacctcattagcttcgg

SEQ ID NO: 237 - DNA, 20 bp, Artificial Sequence
  Antisense Oligonucleotide
ccctctacctcattagcttc

SEQ ID NO: 238 - DNA, 20 bp, Artificial Sequence
  Antisense Oligonucleotide
gacccctctacctcattagc

SEQ ID NO: 239 - DNA, 20 bp, Artificial Sequence
  Antisense Oligonucleotide
gcaaggacccctctacctca

SEQ ID NO: 240 - DNA, 20 bp, Artificial Sequence
  Antisense Oligonucleotide
cagcagcaaggacccctcta

SEQ ID NO: 241 - DNA, 20 bp, Artificial Sequence
  Antisense Oligonucleotide
gagcccagcagcaaggaccc

SEQ ID NO: 242 - DNA, 20 bp, Artificial Sequence
  Antisense Oligonucleotide
tgcacagagcccagcagcaa

SEQ ID NO: 243 - DNA, 20 bp, Artificial Sequence
  Antisense Oligonucleotide
agccctgcacagagcccagc

SEQ ID NO: 244 - DNA, 20 bp, Artificial Sequence
  Antisense Oligonucleotide
catgtagccctgcacagagc

SEQ ID NO: 245 - DNA, 20 bp, Artificial Sequence
  Antisense Oligonucleotide
tgttccatgtagccctgcac

SEQ ID NO: 246 - DNA, 20 bp, Artificial Sequence
  Antisense Oligonucleotide
tggcctgttccatgtagccc

SEQ ID NO: 247 - DNA, 20 bp, Artificial Sequence
  Antisense Oligonucleotide
accttcttggtggcctgttc

SEQ ID NO: 248 - DNA, 20 bp, Artificial Sequence
  Antisense Oligonucleotide
gcgcatcctggaccttcttg

SEQ ID NO: 249 - DNA, 20 bp, Artificial Sequence
  Antisense Oligonucleotide
tgctggttagcgcatcctgg

SEQ ID NO: 250 - DNA, 20 bp, Artificial Sequence
  Antisense Oligonucleotide
ttgcatgctggttagcgcat

SEQ ID NO: 251 - DNA, 20 bp, Artificial Sequence
  Antisense Oligonucleotide
gacttttgcatgctggttag

SEQ ID NO: 252 - DNA, 20 bp, Artificial Sequence
  Antisense Oligonucleotide
cctcagacttttgcatgctg

SEQ ID NO: 253 - DNA, 20 bp, Artificial Sequence
  Antisense Oligonucleotide
agccacctcagacttttgca

SEQ ID NO: 254 - DNA, 20 bp, Artificial Sequence
  Antisense Oligonucleotide
cgcacagccacctcagactt

SEQ ID NO: 255 - DNA, 20 bp, Artificial Sequence
  Antisense Oligonucleotide
ccagtccctggcccgcacag

SEQ ID NO: 256 - DNA, 20 bp, Artificial Sequence
  Antisense Oligonucleotide
gtccatccagtccctggccc

SEQ ID NO: 257 - DNA, 20 bp, Artificial Sequence
  Antisense Oligonucleotide
ccgtccatccagtccctggc

SEQ ID NO: 258 - DNA, 20 bp, Artificial Sequence
  Antisense Oligonucleotide
gccaccgtccatccagtccc

SEQ ID NO: 259 - DNA, 20 bp, Artificial Sequence
  Antisense Oligonucleotide
gtgaagccaccgtccatcca

SEQ ID NO: 260 - DNA, 20 bp, Artificial Sequence
  Antisense Oligonucleotide
ggaggtgaagccaccgtcca

SEQ ID NO: 261 - DNA, 20 bp, Artificial Sequence
  Antisense Oligonucleotide
tgctccagtagcttttcagg

SEQ ID NO: 262 - DNA, 20 bp, Artificial Sequence
  Antisense Oligonucleotide
gtaaatgtgctccagtagct

SEQ ID NO: 263 - DNA, 20 bp, Artificial Sequence
  Antisense Oligonucleotide
tgtcagtaaatgtgctccag

SEQ ID NO: 264 - DNA, 20 bp, Artificial Sequence
  Antisense Oligonucleotide
tggagaccgtgtcagtaaat

SEQ ID NO: 265 - DNA, 20 bp, Artificial Sequence
  Antisense Oligonucleotide
ggctggagaccgtgtcagta

SEQ ID NO: 266 - DNA, 20 bp, Artificial Sequence
  Antisense Oligonucleotide
cagaggctggagaccgtgtc

SEQ ID NO: 267 - DNA, 20 bp, Artificial Sequence
  Antisense Oligonucleotide
atcccagaggctggagaccg

SEQ ID NO: 268 - DNA, 20 bp, Artificial Sequence
  Antisense Oligonucleotide
gaagaatcccagaggctgga

SEQ ID NO: 269 - DNA, 20 bp, Artificial Sequence
  Antisense Oligonucleotide
tctcaaggctcagtagctgg

SEQ ID NO: 270 - DNA, 20 bp, Artificial Sequence
  Antisense Oligonucleotide
tagaggtctcaaggctcagt

SEQ ID NO: 271 - DNA, 20 bp, Artificial Sequence
  Antisense Oligonucleotide
gaacgtagaggtctcaaggc

SEQ ID NO: 272 - DNA, 20 bp, Artificial Sequence
  Antisense Oligonucleotide
catttggaacgtagaggtct

SEQ ID NO: 273 - DNA, 20 bp, Artificial Sequence
  Antisense Oligonucleotide
caagcacatttggaacgtag

SEQ ID NO: 274 - DNA, 20 bp, Artificial Sequence
  Antisense Oligonucleotide
tggacacacaagcacatttg

SEQ ID NO: 275 - DNA, 20 bp, Artificial Sequence
  Antisense Oligonucleotide
caggatggacacacaagcac

SEQ ID NO: 276 - DNA, 20 bp, Artificial Sequence
  Antisense Oligonucleotide
ggccagcaggatggacacac

SEQ ID NO: 277 - DNA, 20 bp, Artificial Sequence
  Antisense Oligonucleotide
gcccagaggccagcaggatg

SEQ ID NO: 278 - DNA, 20 bp, Artificial Sequence
  Antisense Oligonucleotide
cctttcaaacaaccttcagg

SEQ ID NO: 279 - DNA, 20 bp, Artificial Sequence
  Antisense Oligonucleotide
ggacagcatgtttaggtgac

SEQ ID NO: 280 - DNA, 20 bp, Artificial Sequence
  Antisense Oligonucleotide
cttggtggcctgttccatgt

SEQ ID NO: 281 - DNA, 20 bp, Artificial Sequence
  Antisense Oligonucleotide
gttagcgcatcctggacctt

SEQ ID NO: 282 - DNA, 20 bp, Artificial Sequence
  Antisense Oligonucleotide
tggcccgcacagccacctca

SEQ ID NO: 283 - DNA, 20 bp, Artificial Sequence
  Antisense Oligonucleotide
gaccgtgtcagtaaatgtgc

SEQ ID NO: 284 - DNA, 20 bp, Artificial Sequence
  Antisense Oligonucleotide
aagagggcaacaataggagt

SEQ ID NO: 285 - DNA, 20 bp, Artificial Sequence
  Antisense Oligonucleotide
gtgccaagagggcaacaata

SEQ ID NO: 286 - DNA, 20 bp, Artificial Sequence
  Antisense Oligonucleotide
atgccaggagtgccaagagg

SEQ ID NO: 287 - DNA, 20 bp, Artificial Sequence
  Antisense Oligonucleotide
cttcgggcagatgccaggag

SEQ ID NO: 288 - DNA, 20 bp, Artificial Sequence
  Antisense Oligonucleotide
cattagcttcgggcagatgc

SEQ ID NO: 289 - DNA, 20 bp, Artificial Sequence
  Antisense Oligonucleotide
agagcccagcagcaaggacc

SEQ ID NO: 290 - DNA, 20 bp, Artificial Sequence
  Antisense Oligonucleotide
gtggcctgttccatgtagcc

SEQ ID NO: 291 - DNA, 20 bp, Artificial Sequence
  Antisense Oligonucleotide
tcttggtggcctgttccatg

SEQ ID NO: 292 - DNA, 20 bp, Artificial Sequence
  Antisense Oligonucleotide
gaccttcttggtggcctgtt

SEQ ID NO: 293 - DNA, 20 bp, Artificial Sequence
  Antisense Oligonucleotide
tcctggaccttcttggtggc

SEQ ID NO: 294 - DNA, 20 bp, Artificial Sequence
  Antisense Oligonucleotide
ggttagcgcatcctggacct

SEQ ID NO: 295 - DNA, 20 bp, Artificial Sequence
  Antisense Oligonucleotide
atgctggttagcgcatcctg

SEQ ID NO: 296 - DNA, 20 bp, Artificial Sequence
  Antisense Oligonucleotide
tttgcatgctggttagcgca

SEQ ID NO: 297 - DNA, 20 bp, Artificial Sequence
  Antisense Oligonucleotide
agacttttgcatgctggtta

SEQ ID NO: 298 - DNA, 20 bp, Artificial Sequence
  Antisense Oligonucleotide
acctcagacttttgcatgct

SEQ ID NO: 299 - DNA, 20 bp, Artificial Sequence
  Antisense Oligonucleotide
cagccacctcagacttttgc

SEQ ID NO: 300 - DNA, 20 bp, Artificial Sequence
  Antisense Oligonucleotide
ccgcacagccacctcagact

SEQ ID NO: 301 - DNA, 20 bp, Artificial Sequence
  Antisense Oligonucleotide
ctggcccgcacagccacctc

SEQ ID NO: 302 - DNA, 20 bp, Artificial Sequence
  Antisense Oligonucleotide
agtccctggcccgcacagcc

SEQ ID NO: 303 - DNA, 20 bp, Artificial Sequence
  Antisense Oligonucleotide
catccagtccctggcccgca

SEQ ID NO: 304 - DNA, 20 bp, Artificial Sequence
  Antisense Oligonucleotide
agccaccgtccatccagtcc

SEQ ID NO: 305 - DNA, 20 bp, Artificial Sequence
  Antisense Oligonucleotide
ggtgaagccaccgtccatcc

SEQ ID NO: 306 - DNA, 20 bp, Artificial Sequence
  Antisense Oligonucleotide
agggaggtgaagccaccgtc

SEQ ID NO: 307 - DNA, 20 bp, Artificial Sequence
  Antisense Oligonucleotide
ttttcagggaggtgaagcca

SEQ ID NO: 308 - DNA, 20 bp, Artificial Sequence
  Antisense Oligonucleotide
agtagcttttcagggaggtg

SEQ ID NO: 309 - DNA, 20 bp, Artificial Sequence
  Antisense Oligonucleotide
aaatgtgctccagtagcttt

SEQ ID NO: 310 - DNA, 20 bp, Artificial Sequence
  Antisense Oligonucleotide
tcagtaaatgtgctccagta

SEQ ID NO: 311 - DNA, 20 bp, Artificial Sequence
  Antisense Oligonucleotide
ccgtgtcagtaaatgtgctc

SEQ ID NO: 312 - DNA, 20 bp, Artificial Sequence
  Antisense Oligonucleotide
ggagaccgtgtcagtaaatg

SEQ ID NO: 313 - DNA, 20 bp, Artificial Sequence
  Antisense Oligonucleotide
aggctggagaccgtgtcagt

SEQ ID NO: 314 - DNA, 20 bp, Artificial Sequence
  Antisense Oligonucleotide
cccagaggctggagaccgtg

SEQ ID NO: 315 - DNA, 20 bp, Artificial Sequence
  Antisense Oligonucleotide
agaatcccagaggctggaga

SEQ ID NO: 316 - DNA, 20 bp, Artificial Sequence
  Antisense Oligonucleotide
agaggtctcaaggctcagta

SEQ ID NO: 317 - DNA, 20 bp, Artificial Sequence
  Antisense Oligonucleotide
aacgtagaggtctcaaggct

SEQ ID NO: 318 - DNA, 20 bp, Artificial Sequence
  Antisense Oligonucleotide
tttggaacgtagaggtctca

SEQ ID NO: 319 - DNA, 20 bp, Artificial Sequence
  Antisense Oligonucleotide
gcacatttggaacgtagagg

SEQ ID NO: 320 - DNA, 20 bp, Artificial Sequence
  Antisense Oligonucleotide
cacaagcacatttggaacgt

SEQ ID NO: 321 - DNA, 20 bp, Artificial Sequence
  Antisense Oligonucleotide
ggacacacaagcacatttgg

SEQ ID NO: 322 - DNA, 20 bp, Artificial Sequence
  Antisense Oligonucleotide
aggatggacacacaagcaca

SEQ ID NO: 323 - DNA, 20 bp, Artificial Sequence
  Antisense Oligonucleotide
ccagcaggatggacacacaa

SEQ ID NO: 324 - DNA, 20 bp, Artificial Sequence
  Antisense Oligonucleotide
agaggccagcaggatggaca

SEQ ID NO: 325 - DNA, 20 bp, Artificial Sequence
  Antisense Oligonucleotide
ggcccagaggccagcaggat

SEQ ID NO: 326 - DNA, 20 bp, Artificial Sequence
  Antisense Oligonucleotide
acccaggcccagaggccagc

SEQ ID NO: 327 - DNA, 20 bp, Artificial Sequence
  Antisense Oligonucleotide
gggccacccaggcccagagg

SEQ ID NO: 328 - DNA, 20 bp, Artificial Sequence
  Antisense Oligonucleotide
ctttccctttcaaacaacct

SEQ ID NO: 329 - DNA, 20 bp, Artificial Sequence
  Antisense Oligonucleotide
caatactttccctttcaaac

SEQ ID NO: 330 - DNA, 20 bp, Artificial Sequence
  Antisense Oligonucleotide
catgacaatactttcccttt

SEQ ID NO: 331 - DNA, 20 bp, Artificial Sequence
  Antisense Oligonucleotide
gaaaacatgacaatactttc

SEQ ID NO: 332 - DNA, 20 bp, Artificial Sequence
  Antisense Oligonucleotide
gggatgaaaacatgacaata

SEQ ID NO: 333 - DNA, 20 bp, Artificial Sequence
  Antisense Oligonucleotide
catgtttaggtgacttctgg

SEQ ID NO: 334 - DNA, 20 bp, Artificial Sequence
  Antisense Oligonucleotide
gacagcatgtttaggtgact

SEQ ID NO: 335 - DNA, 20 bp, Artificial Sequence
  Antisense Oligonucleotide
tttaggacagcatgtttagg

SEQ ID NO: 336 - DNA, 20 bp, Artificial Sequence
  Antisense Oligonucleotide
ctttatttaggacagcatgt

SEQ ID NO: 337 - DNA, 20 bp, Artificial Sequence
  Antisense Oligonucleotide
tccagctttatttaggacag

(19) United States
(12) Patent Application Publication
(10) Pub. No.: US 2015/0045431 A1
(43) Pub. Date: Feb. 12, 2015

(54) METHODS OF TREATING A CARDIOVASCULAR DISORDER IN A SUBJECT ON APO-C3 MODULATING THERAPY

(71) Applicant: Amarin Pharmaceuticals Ireland Limited, Dublin (IE)

(72) Inventor: Joseph S. Zakrzewski, Bedminster, NJ (US)

(21) Appl. No.: 14/453,167

(22) Filed: Aug. 6, 2014

Related U.S. Application Data
(60) Provisional application No. 61/862,577, filed on Aug. 6, 2013.

(51) Int. Cl. A61K 31/557

(52) U.S. Cl. CPC A61K 31/557
USPC 514/549

(57) ABSTRACT

In various embodiments, the present invention provides methods of treating and/or preventing cardiovascular-related disease and, in particular, a method of reducing triglycerides in a subject on Apo-C3 modulating therapy, the method comprising administering to a subject in need thereof a pharmaceutical composition comprising eicosapentaenoic acid or a derivative thereof.

### PRIORITY CLAIM

The present application claims priority to U.S. provisional patent application Ser. No. 61/862,577, filed on Aug. 6, 2013, the entire contents of which are incorporated herein by reference and relied upon.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dyslipidemia, congestive heart failure and stroke. A need exists for improved treatments for cardiovascular diseases and disorders.

### SUMMARY

In various embodiments, the present invention provides methods of treating and/or preventing cardiovascular-related diseases and, in particular, a method of treating hypercholesterolemia comprising administering to a subject in need thereof a pharmaceutical composition comprising eicosapentaenoic acid or a derivative thereof. In one embodiment, the composition is co-administered with an apolipoprotein C-III (“Apo-C3”) modulating compound. In one embodiment, the Apo-C3 modulating compound modulates the expression of Apo-C3. In one embodiment, the Apo-C3 modulating compound comprises an oligonucleotide. In one embodiment, the oligonucleotide targets nucleic acid encoding Apo-C3.

In one embodiment, the composition contains not more than 10%, by weight, of all fatty acids (and/or derivatives thereof) present, docosahexaenoic acid or derivative thereof, substantially no docosahexaenoic acid or derivative thereof, or no docosahexaenoic acid or derivative thereof. In another embodiment, eicosapentaenoic acid ethyl ester comprises at least 96%, by weight, of all fatty acids (and/or derivatives thereof) present in the composition; the composition contains not more than 4%, by weight, of total fatty acids (and/or derivatives thereof) other than eicosapentaenoic acid ethyl ester; and/or the composition contains about 0.1% to about 0.6% of at least one fatty acid other than eicosapentaenoic acid ethyl ester and docosahexaenoic acid (or derivative thereof).

In one embodiment, a pharmaceutical composition useful in accordance with the invention comprises, consists of or consists essentially of at least 95%, by weight of all fatty acids (and/or derivatives thereof) present, ethyl eicosapentaenoate (EPA-E), about 0.2% to about 0.5%, by weight of all fatty acids (and/or derivatives thereof) present, ethyl octadecatetraenoate (ODTA-E), about 0.05% to about 0.25%, by weight of all fatty acids (and/or derivatives thereof) present, ethyl nonaecapentaenoate (NDPA-E), about 0.2% to about 0.45%, by weight of all fatty acids (and/or derivatives thereof) present, ethyl arachidonate (AA-E), about 0.3% to about 0.5%, by weight of all fatty acids (and/or derivatives thereof) present, ethyl eicosatetraenoate (ETA-E), and about 0.05% to about 0.32%, by weight of all fatty acids (and/or derivatives thereof) present, ethyl heneicosapentaenoate (HPA-E). In another embodiment, the composition is present in a capsule shell. In another embodiment, the composition contains substantially no or no amount of docosahexaenoic acid (DHA) or derivative thereof such as ethyl-DHA (DHA-E).

In another embodiment, the invention provides a method of treating moderate to severe hypertriglyceridemia comprising administering a composition as described herein to a subject in need thereof one to about four times per day.

In some embodiments, a method of reducing triglycerides in a subject on Apo-C3 modulating therapy according to the present invention comprises administering to the subject a composition comprising at least about 80%, by weight of all fatty acids (and/or derivatives thereof) present, ethyl eicosapentaenoate.

These and other embodiments of the present invention will be disclosed in further detail herein below.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

In one embodiment, the invention provides a method for treatment and/or prevention of a cardiovascular-related disease. The term “cardiovascular-related disease” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the present invention provides a method of blood lipid therapy comprising administering to a subject or subject group in need thereof a pharmaceutical composition as described herein. In another embodiment, the subject or subject group has one or more of: hypercholesterolemia, familial hypercholesterolemia, high LDL-C serum levels, high total cholesterol levels, and/or low HDL-C serum levels.

In another embodiment, the subject or subject group being treated has a baseline triglyceride level (or median baseline triglyceride level in the case of a subject group), fed or fasting, of at least about 300 mg/dl, at least about 400 mg/dl, at least about 500 mg/dl, at least about 600 mg/dl, at least about 700 mg/dl, at least about 800 mg/dl, at least about 900 mg/dl, at least about 1000 mg/dl, at least about 1100 mg/dl, at least about 1200 mg/dl, at least about 1300 mg/dl, at least about 1400 mg/dl, or at least about 1500 mg/dl, for example about 400 mg/dl to about 2500 mg/dl, about 450 mg/dl to about 2000 mg/dl or about 500 mg/dl to about 1500 mg/dl.

In one embodiment, the subject or subject group being treated in accordance with methods of the invention has previously been treated with Lovaza® and has experienced an increase in, or no decrease in, LDL-C levels and/or non-HDL-C levels. In one such embodiment, Lovaza® therapy is discontinued and replaced by a method of the present invention.

In another embodiment, the subject or subject group being treated in accordance with methods of the invention exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the invention exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2% or not more than about 0.15%. In one such embodiment, free plasma EPA and/or total fatty acid levels are determined prior to initiating therapy.

In another embodiment, the subject or subject group being treated in accordance with methods of the invention exhibits a fasting baseline absolute plasma level of total fatty acid (or mean thereof) not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the invention exhibits a fasting baseline plasma, serum or red blood cell membrane EPA level not greater than about 70 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present invention comprise a step of measuring the subject's (or subject group's mean) baseline lipid profile prior to initiating therapy. In another embodiment, methods of the invention comprise the step of identifying a subject or subject group having one or more of the following: baseline non-HDL-C value of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value of about 250 mg/dl to about 400 mg/dl, for example at least about 260 mg/dl, at least about 270 mg/dl, at least about 280 mg/dl or at least about 290 mg/dl; baseline vLDL-C value of about 140 mg/dl to about 200 mg/dl, for example at least about 150 mg/dl, at least about 160 mg/dl, at least about 170 mg/dl, at least about 180 mg/dl or at least about 190 mg/dl; baseline HDL-C value of about 10 to about 60 mg/dl, for example not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value of about 50 to about 300 mg/dl, for example not less than about 100 mg/dl, not less than about 90 mg/dl, not less than about 80 mg/dl, not less than about 70 mg/dl, not less than about 60 mg/dl or not less than about 50 mg/dl.

In a related embodiment, upon treatment in accordance with the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits one or more of the following outcomes:

(a) reduced triglyceride levels compared to baseline or control;

(b) reduced Apo B levels compared to baseline or control;

(c) increased HDL-C levels compared to baseline or control;

(d) no increase in LDL-C levels compared to baseline or control;

(e) a reduction in LDL-C levels compared to baseline or control;

(f) a reduction in non-HDL-C levels compared to baseline or control;

(g) a reduction in vLDL levels compared to baseline or control;

(h) an increase in apo A-I levels compared to baseline or control;

(i) an increase in apo A-I/apo B ratio compared to baseline or control;

(j) a reduction in lipoprotein A levels compared to baseline or control;

(k) a reduction in LDL particle number compared to baseline or control;

(l) an increase in LDL size compared to baseline or control;

(m) a reduction in remnant-like particle cholesterol compared to baseline or control;

(n) a reduction in oxidized LDL compared to baseline or control;

(o) no change or a reduction in fasting plasma glucose (FPG) compared to baseline or control;

(p) a reduction in hemoglobin A1c (HbA1c) compared to baseline or control;

(q) a reduction in homeostasis model insulin resistance compared to baseline or control;

(r) a reduction in lipoprotein associated phospholipase A2 compared to baseline or control;

(s) a reduction in intracellular adhesion molecule-1 compared to baseline or control;

(t) a reduction in interleukin-6 compared to baseline or control;

(u) a reduction in plasminogen activator inhibitor-1 compared to baseline or control;

(v) a reduction in high sensitivity C-reactive protein (hsCRP) compared to baseline or control;

(w) an increase in serum or plasma EPA compared to baseline or control;

(x) an increase in red blood cell (RBC) membrane EPA compared to baseline or control;

(y) a reduction or increase in one or more of serum phospholipid and/or red blood cell content of docosahexaenoic acid (DHA), docosapentaenoic acid (DPA), arachidonic acid (AA), palmitic acid (PA), staeridonic acid (SA) or oleic acid (OA) compared to baseline or control;

(z) a reduction in or prevention of membrane cholesterol domain formation compared to baseline or control; and/or

(aa) a reduction in or prevention of oxidative modification of membrane polyunsaturated fatty acids compared to baseline or control.

In one embodiment, upon administering a composition of the invention to a subject, the subject exhibits a decrease in triglyceride levels, an increase in the concentrations of EPA and DPA (n-3) in red blood cells, and an increase of the ratio of EPA:arachidonic acid in red blood cells. In a related embodiment the subject exhibits substantially no or no increase in RBC DHA.

In one embodiment, methods of the present invention comprise measuring baseline levels of one or more markers set forth in (a)-(aa) above prior to dosing the subject or subject group. In another embodiment, the methods comprise administering a composition as disclosed herein to the subject after baseline levels of one or more markers set forth in (a)-(aa) are determined, and subsequently taking an additional measurement of said one or more markers.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more, any 24 or more, any 25 or more, any 26 or more, or all 27 of outcomes (a)-(aa) described immediately above.

In another embodiment, upon treatment with a composition of the present invention, the subject or subject group exhibits one or more of the following outcomes:

(a) a reduction in triglyceride level of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(b) a less than 30% increase, less than 20% increase, less than 10% increase, less than 5% increase or no increase in non-HDL-C levels or a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(c) substantially no change in HDL-C levels, no change in HDL-C levels, or an increase in HDL-C levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(d) a less than 60% increase, a less than 50% increase, a less than 40% increase, a less than 30% increase, less than 20% increase, less than 10% increase, less than 5% increase or no increase in LDL-C levels or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55% or at least about 75% (actual % change or median % change) as compared to baseline;

(e) a decrease in Apo B levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(f) a reduction in vLDL levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(g) an increase in apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(h) an increase in apo A-I/apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(i) a reduction in lipoprotein (a) levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(j) a reduction in mean LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(k) an increase in mean LDL particle size of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(l) a reduction in remnant-like particle cholesterol of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(m) a reduction in oxidized LDL of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(n) substantially no change, no significant change, or a reduction (e.g. in the case of a diabetic subject) in fasting plasma glucose (FPG) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(o) substantially no change, no significant change or a reduction in hemoglobin A1c (HbA1c) of at least about 5%, at least about 10%, at least about 15%, at least about 20%, at least about 25%, at least about 30%, at least about 35%, at least about 40%, at least about 45%, or at least about 50% (actual % change or median % change) compared to baseline;

(p) a reduction in homeostasis model index insulin resistance of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(q) a reduction in lipoprotein associated phospholipase A2 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(r) a reduction in intracellular adhesion molecule-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(s) a reduction in interleukin-6 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(t) a reduction in plasminogen activator inhibitor-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(u) a reduction in high sensitivity C-reactive protein (hsCRP) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(v) an increase in serum, plasma and/or RBC EPA of at least about 5%, at least about 10%, at least about 15%, at least about 20%, at least about 25%, at least about 30%, at least about 35%, at least about 40%, at least about 45%, at least about 50%, at least about 100%, at least about 200% or at least about 400% (actual % change or median % change) compared to baseline;

(w) an increase in serum phospholipid and/or red blood cell membrane EPA of at least about 5%, at least about 10%, at least about 15%, at least about 20%, at least about 25%, at least about 30%, at least about 35%, at least about 40%, at least about 45%, r at least about 50%, at least about 100%, at least about 200%, or at least about 400% (actual % change or median % change) compared to baseline;

(x) a reduction or increase in one or more of serum phospholipid and/or red blood cell DHA, DPA, AA, PA and/or OA of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline;

(y) a reduction in total cholesterol of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline;

(z) a reduction in membrane cholesterol domain formation of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or about 100% (actual % change or median % change) compared to baseline or control; and/or

(aa) a reduction in oxidative modification of membrane polyunsaturated fatty acids of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or about 100% (actual % change or median % change) compared to baseline or control.

In one embodiment, methods of the present invention comprise measuring baseline levels of one or more markers set forth in (a)-(aa) prior to dosing the subject or subject group. In another embodiment, the methods comprise administering a composition as disclosed herein to the subject after baseline levels of one or more markers set forth in (a)-(aa) are determined, and subsequently taking a second measurement of the one or more markers as measured at baseline for comparison thereto.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, any 25 or more of, any 26 or more of, or all 27 of outcomes (a)-(aa) described immediately above.

Parameters (a)-(y) can be measured in accordance with any clinically acceptable methodology. For example, triglycerides, total cholesterol, HDL-C and fasting blood sugar can be sample from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnants lipoproteins and LDL-phospholipase A2 can be determined from EDTA plasma or serum and serum, respectively, using enzymatic immunoseparation techniques. Oxidized LDL, intercellular adhesion molecule-1 and interleukin-6 levels can be determined from serum using standard enzyme immunoassay techniques. These techniques are described in detail in standard textbooks, for example Tietz Fundamentals of Clinical Chemistry, 6th Ed. (Burtis, Ashwood and Borter Eds.), WB Saunders Company. Parameters (z) and (aa) can be measured in accordance with any clinically acceptable methodology or can be estimated by any suitable in vitro experiment, for example, one similar to that described in Example 3.

In one embodiment, subjects fast for up to 12 hours prior to blood sample collection, for example about 10 hours.

In another embodiment, the present invention provides a method of treating or preventing primary hypercholesterolemia and/or mixed dyslipidemia (Fredrickson Types IIa and IIb) in a patient in need thereof, comprising administering to the patient one or more compositions as disclosed herein. In a related embodiment, the present invention provides a method of reducing triglyceride levels in a subject or subjects when treatment with a statin or niacin extended-release monotherapy is considered inadequate (Frederickson type IV hyperlipidemia).

In another embodiment, the present invention provides a method of treating or preventing risk of recurrent nonfatal myocardial infarction in a patient with a history of myocardial infarction, comprising administering to the patient one or more compositions as disclosed herein.

In another embodiment, the present invention provides a method of slowing progression of or promoting regression of atherosclerotic disease in a patient in need thereof, comprising administering to a subject in need thereof one or more compositions as disclosed herein.

In another embodiment, the present invention provides a method of treating or preventing very high serum triglyceride levels (e.g. Types IV and V hyperlipidemia) in a patient in need thereof, comprising administering to the patient one or more compositions as disclosed herein.

In another embodiment, the present invention provides a method of treating subjects having very high serum triglyceride levels (e.g. greater than 1000 mg/dl or greater than 2000 mg/dl) and that are at risk of developing pancreatitis, comprising administering to the patient one or more compositions as disclosed herein.

In one embodiment, a composition of the invention is administered to a subject in an amount sufficient to provide a daily dose of eicosapentaenoic acid of about 1 mg to about 10,000 mg, 25 about 5000 mg, about 50 to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In another embodiment, any of the methods disclosed herein are used in treatment or prevention of a subject or subjects that consume a traditional Western diet. In one embodiment, the methods of the invention include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60% or more or 70% of total calories come from these sources.

In one embodiment, a composition for use in methods of the invention comprises eicosapentaenoic acid, or a pharmaceutically acceptable ester, derivative, conjugate or salt thereof, or mixtures of any of the foregoing, collectively referred to herein as “EPA.” The term “pharmaceutically acceptable” in the present context means that the substance in question does not produce unacceptable toxicity to the subject or interaction with other components of the composition.

In one embodiment, the EPA comprises all-cis eicosa-5,8,11,14,17-pentaenoic acid. In another embodiment, the EPA comprises an eicosapentaenoic acid ester. In another embodiment, the EPA comprises a C1-C5 alkyl ester of eicosapentaenoic acid. In another embodiment, the EPA comprises eicosapentaenoic acid ethyl ester, eicosapentaenoic acid methyl ester, eicosapentaenoic acid propyl ester, or eicosapentaenoic acid butyl ester. In another embodiment, the EPA comprises In one embodiment, the EPA comprises all-cis eicosa-5,8,11,14,17-pentaenoic acid ethyl ester.

In another embodiment, the EPA is in the form of ethyl-EPA, lithium EPA, mono-, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree.

In another embodiment, EPA is present in a composition useful in accordance with methods of the invention in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg

In another embodiment, a composition useful in accordance with the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of all fatty acids (and/or derivatives thereof) present, docosahexaenoic acid (DHA), if any. In another embodiment, a composition of the invention contains substantially no docosahexaenoic acid. In still another embodiment, a composition useful in the present invention contains no docosahexaenoic acid and/or derivative thereof.

In another embodiment, EPA comprises at least 70%, at least 80%, at least 90%, at least 95%, at least 96%, at least 97%, at least 98%, at least 99%, or 100%, by weight of all fatty acids (and/or derivatives thereof) present, in a composition that is useful in methods of the present invention.

In one embodiment, a composition of the invention comprises ultra-pure EPA. The term “ultra-pure” as used herein with respect to EPA refers to a composition comprising at least 95%, by weight of all fatty acids (and/or derivatives thereof) present, EPA (as the term “EPA” is defined and exemplified herein). Ultra-pure EPA comprises at least 96%, by weight of all fatty acids (and/or derivatives thereof) present, EPA, at least 97%, by weight of all fatty acids (and/or derivatives thereof) present, EPA, or at least 98%, by weight of all fatty acids (and/or derivatives thereof) present, EPA, wherein the EPA is any form of EPA as set forth herein.

In another embodiment, a composition useful in accordance with methods of the invention contains less than 10%, less than 9%, less than 8%, less than 7%, less than 6%, less than 5%, less than 4%, less than 3%, less than 2%, less than 1%, less than 0.5% or less than 0.25%, by weight of all fatty acids (and/or derivatives thereof) present, of any fatty acid other than EPA. Illustrative examples of a “fatty acid other than EPA” include linolenic acid (LA), arachidonic acid (AA), docosahexaenoic acid (DHA), alpha-linolenic acid (ALA), stearadonic acid (STA), eicosatrienoic acid (ETA) and/or docosapentaenoic acid (DPA). In another embodiment, a composition useful in accordance with methods of the invention contains about 0.1% to about 4%, about 0.5% to about 3%, or about 1% to about 2%, by weight of all fatty acids (and/or derivatives thereof) present, other than EPA and/or DHA.

In another embodiment, a composition useful in accordance with the invention has one or more of the following features: (a) eicosapentaenoic acid ethyl ester represents at least about 96%, at least about 97%, or at least about 98%, by weight of all fatty acids (and/or derivatives thereof) present, in the composition; (b) the composition contains not more than about 4%, not more than about 3%, or not more than about 2%, by weight of all fatty acids (and/or derivatives thereof) present, other than eicosapentaenoic acid ethyl ester; (c) the composition contains not more than about 0.6%, not more than about 0.5%, or not more than about 0.4%, by weight of all fatty acids (and/or derivatives thereof) present, of any individual fatty acid other than eicosapentaenoic acid ethyl ester; (d) the composition has a refractive index (20° C.) of about 1 to about 2, about 1.2 to about 1.8 or about 1.4 to about 1.5; (e) the composition has a specific gravity (20° C.) of about 0.8 to about 1.0, about 0.85 to about 0.95 or about 0.9 to about 0.92; (e) the composition contains not more than about 20 ppm, not more than about 15 ppm or not more than about 10 ppm heavy metals, (f) the composition contains not more than about 5 ppm, not more than about 4 ppm, not more than about 3 ppm, or not more than about 2 ppm arsenic, and/or (g) the composition has a peroxide value of not more than about 5 meq/kg, not more than about 4 meq/kg, not more than about 3 meq/kg, or not more than about 2 meq/kg.

In another embodiment, a composition useful in accordance with the invention comprises, consists of or consists essentially of at least 95%, by weight of all fatty acids (and/or derivatives thereof) present, ethyl eicosapentaenoate (EPA-E), about 0.2% to about 0.5%, by weight of all fatty acids (and/or derivatives thereof) present, ethyl octadecatetraenoate (ODTA-E), about 0.05% to about 0.25%, by weight of all fatty acids (and/or derivatives thereof) present, ethyl nonaecapentaenoate (NDPA-E), about 0.2% to about 0.45%, by weight of all fatty acids (and/or derivatives thereof) present, ethyl arachidonate (AA-E), about 0.3% to about 0.5%, by weight of all fatty acids (and/or derivatives thereof) present, ethyl eicosatetraenoate (ETA-E), and about 0.05% to about 0.32%, by weight of all fatty acids (and/or derivatives thereof) present, ethyl heneicosapentaenoate (HPA-E). In another embodiment, the composition is present in a capsule shell.

In another embodiment, compositions useful in accordance with the invention comprise, consist essential of, or consist of at least 95%, 96% or 97%, by weight of all fatty acids (and/or derivatives thereof) present, ethyl eicosapentaenoate, about 0.2% to about 0.5% by weight ethyl octadecatetraenoate, about 0.05% to about 0.25%, by weight of all fatty acids (and/or derivatives thereof) present, ethyl nonaecapentaenoate, about 0.2% to about 0.45%, by weight of all fatty acids (and/or derivatives thereof) present, ethyl arachidonate, about 0.3% to about 0.5%, by weight of all fatty acids (and/or derivatives thereof) present, ethyl eicosatetraenoate, and about 0.05% to about 0.32%, by weight of all fatty acids (and/or derivatives thereof) present, ethyl heneicosapentaenoate. Optionally, the composition contains not more than about 0.06%, about 0.05%, or about 0.04%, by weight of all fatty acids (and/or derivatives thereof) present, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell.

In another embodiment, compositions useful in accordance with the invention comprise, consist essential of, or consist of at least 96%, by weight of all fatty acids (and/or derivatives thereof) present, ethyl eicosapentaenoate, about 0.22% to about 0.4%, by weight of all fatty acids (and/or derivatives thereof) present, ethyl octadecatetraenoate, about 0.075% to about 0.20%, by weight of all fatty acids (and/or derivatives thereof) present, ethyl nonaecapentaenoate, about 0.25% to about 0.40%, by weight of all fatty acids (and/or derivatives thereof) present, ethyl arachidonate, about 0.3% to about 0.4%, by weight of all fatty acids (and/or derivatives thereof) present, ethyl eicosatetraenoate and about 0.075% to about 0.25%, by weight of all fatty acids (and/or derivatives thereof) present, ethyl heneicosapentaenoate. Optionally, the composition contains not more than about 0.06%, about 0.05%, or about 0.04%, by weight of all fatty acids (and/or derivatives thereof) present, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell. In one embodiment, the dosage form is a gel or liquid capsule and is packaged in blister packages of about 1 to about 20 capsules per sheet.

In another embodiment, compositions useful in accordance with the invention comprise, consist essential of, or consist of at least 96%, 97% or 98%, by weight of all fatty acids (and/or derivatives thereof) present, ethyl eicosapentaenoate, about 0.25% to about 0.38%, by weight of all fatty acids (and/or derivatives thereof) present, ethyl octadecatetraenoate, about 0.10% to about 0.15%, by weight of all fatty acids (and/or derivatives thereof) present, ethyl nonaecapentaenoate, about 0.25% to about 0.35%, by weight of all fatty acids (and/or derivatives thereof) present, ethyl arachidonate, about 0.31% to about 0.38%, by weight of all fatty acids (and/or derivatives thereof) present, ethyl eicosatetraenoate, and about 0.08% to about 0.20%, by weight of all fatty acids (and/or derivatives thereof) present, ethyl heneicosapentaenoate. Optionally, the composition contains not more than about 0.06%, about 0.05%, or about 0.04%, by weight of all fatty acids (and/or derivatives thereof) present, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell.

In another embodiment, a composition as described herein is administered to a subject once or twice per day. In another embodiment, 1, 2, 3 or 4 capsules, each containing about 1 g of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 pm and about 11 pm.

In one embodiment, a subject being treated in accordance with methods of the invention is not otherwise on lipid-altering therapy, for example statin, fibrate, niacin and/or ezetimibe therapy.

In another embodiment, compositions useful in accordance with methods of the invention are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

In some embodiments, a composition of the present disclosure comprises eicosapentaenoic acid or a derivative thereof and an Apo-C3 modulating compound. As used herein, the term “Apo-C3 modulating compound” refers to a compound that modulates the expression of Apo-C3. In one embodiment, the Apo-C3 modulating compound comprises an oligonucleotide. In one embodiment, the oligonucleotide targets nucleic acid encoding Apo-C3.

Apo-C3 is a component of HDL and of triglyceride-rich lipoproteins, and plays a role in hypertriglyceridemia. Apo-C3 has been shown to slow the clearance of triglyceride-rich lipoproteins by inhibiting lipolysis in two ways: by inhibiting lipoprotein lipase, and by interfering the binding of lipoprotein to the cell-surface glycosaminoglycan matrix.

In some embodiments, the Apo-C3 modulating compound is a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof, as well as modified oligonucleotides in which a different nucleobase is present at one or more of the nucleotide positions in the oligonucleotide. Modification of an oligonucleotide may be done at any of the positions of the oligonucleotide. The term “oligonucleotide” also includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

The Apo-C3 modulating compounds may comprise from about 8 to about 80 nucleobases (i.e. from about 8 to about 80 linked nucleosides), for example 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

Antisense compounds 8-80 nucleobases in length comprising a stretch of at least eight (8) consecutive nucleobases selected from within the illustrative antisense compounds are considered to be suitable antisense compounds as well.

Exemplary preferred antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). Exemplary compounds of this invention from a variety of mammalian sources, including human, may be found identified in Table 1.

TABLE 1
Example Apo-C3 Modulating Compounds.

| SEQ ID NO | SEQUENCE |
|---|---|
| 1 | ctggagcagctgcctctagg |
| 2 | ccctgcatgaagctgagaag |
| 3 | gtgcttcatgtaaccctgca |
| 4 | tggcctgctgggccacctgg |
| 5 | tgctccagtagtctttcagg |
| 6 | tgacctcagggtccaaatcc |
| 7 | ctctagggatgaactgagca |
| 8 | cagctgcctctagggatgaa |
| 9 | ttcctggagcagctgcctct |
| 10 | acctctgttcctggagcagc |
| 11 | atggcacctctgttcctgga |
| 12 | gggctgcatggcacctctgt |
| 13 | ggcaacaacaaggagtaccc |
| 14 | ggagggcaacaacaaggagt |
| 15 | agctcgggcagaggccagga |
| 16 | tctgaagctcgggcagaggc |
| 17 | cggcctctgaagctcgggca |
| 18 | catcctcggcctctgaagct |
| 19 | gggaggcatcctcggcctct |
| 20 | gagaagggaggcatcctcgg |
| 21 | gctgagaagggaggcatcct |
| 22 | tgcatgaagctgagaaggga |
| 23 | gcgtgcttcatgtaaccctg |
| 24 | ttggtggcgtgcttcatgta |
| 25 | gcatccttggcggtcttggt |
| 26 | ctcagtgcatccttggcggt |
| 27 | tgctcagtgcatccttggcy |
| 28 | ctcctgcacgctgctcagtg |
| 29 | gactcctgcacgctgctcag |
| 30 | gccacctgggactcctgcac |
| 31 | gcccctggcctgctgggcca |
| 32 | agcccctggcctgctgggcc |
| 33 | gaagccatcggtcacccagc |
| 34 | ctgaagccatcggtcaccca |
| 35 | tttcagggaactgaagccat |
| 36 | cagtagtctttcagggaact |
| 37 | aacggtgctccagtagtctt |
| 38 | ccttaacggtgctccagtag |
| 39 | gaacttgtccttaacggtgc |
| 40 | ctcagagaacttgtccttaa |
| 41 | agaactcagagaacttgtcc |
| 42 | atcccagaactcagagaact |
| 43 | cagggtccaaatcccagaac |
| 44 | ttggtctgacctcagggtcc |
| 45 | gttggtctgacctcagggtc |
| 46 | gctgaagttggtctgacctc |
| 47 | cagccacggctgaagttggt |
| 48 | caggcagccacggctgaagt |
| 49 | attgaggtctcaggcagcca |
| 50 | tggataggcaggtggacttg |
| 51 | ctcgcaggatggataggcag |
| 52 | aggagctcgcaggatggata |
| 53 | gacccaaggagctcgcagga |
| 54 | tgcaggacccaaggagctcg |
| 55 | tggagattgcaggacccaag |
| 56 | agccctggagattgcaggac |
| 57 | ggcagccctggagattgcag |
| 58 | ccttttaagcaacctacagg |
| 59 | ctgtcccttttaagcaacct |
| 60 | agaatactgtcccttttaag |
| 61 | cactgagaatactgtccctt |
| 62 | taggagagcactgagaatac |
| 63 | gggtaggagagcactgagaa |
| 64 | aggccagcatgcctggaggg |
| 65 | ttgggaggccagcatgcctg |
| 66 | agctttattgggaggccagc |
| 67 | tgtccagctttattgggagg |
| 68 | cttgtccagctttattggga |
| 69 | agcttcttgtccagctttat |
| 70 | catagcagcttcttgtccag |
| 71 | acctggagcagctgcctcta |
| 72 | agggcattacctggagcagc |
| 73 | acctctgttcctgcaaggaa |
| 74 | aagtgcttacgggcagaggc |
| 75 | gcgggtgtacctggcctgct |
| 76 | aaccctttgtgaactgcac |
| 77 | agtgagcaataccgcctgag |
| 78 | cggcttgaattaggtcagg |
| 79 | tagggataaaactgagcagg |
| 80 | ctggagtagctagctgcttc |
| 81 | gctgcatggcacctacgtac |
| 82 | ccacagtgaggagcgtccgg |
| 83 | ggcagatgccaggagagcca |
| 84 | ctacctcttcagctcgggca |
| 85 | cagcagcaaggatccctcta |
| 86 | gcacagagcccagcagcaag |
| 87 | ccctggccaccgcagctata |
| 88 | atctgaagtgattgtccatc |
| 89 | agtagcctttcaggaatctg |
| 90 | cttgtcagtaaacttgctcc |
| 91 | gaagccggtgaacttgtcag |
| 92 | gaatcccagaagccggtgaa |
| 93 | ggttagaatcccagaagccg |
| 94 | tggagttggttggtcctcag |
| 95 | tcacgactcaatagctggag |
| 96 | cccttaaagcaaccttcagg |
| 97 | agacatgagaacatactttc |
| 98 | catgtttaggtgagatctag |
| 99 | tcttatccagctttattagg |
| 100 | gctgcatggcacctctgttc |
| 101 | ggcagaggccaggagcgcca |
| 102 | ctgaagctcgggcagaggcc |
| 103 | tcctcggcctctgaagctcg |
| 104 | tcttggtggcgtgcttcatg |
| 105 | gctcagtgcatccttggcgg |
| 106 | cctgcacgctgctcagtgca |
| 107 | actgaagccatcggtcaccc |
| 108 | cagaactcagagaacttgtc |
| 109 | gaagttggtctgacctcagg |
| 110 | ccctggagattgcaggaccc |
| 111 | gggcagccctggagattgca |
| 112 | cccttttaagcaacctacag |
| 113 | TGAACTTATCAGTGAACTTG |
| 114 | TCAGGGCCAGACTCCCAGAG |
| 115 | TTGGTGTTGTTAGTTGGTCC |
| 116 | TTGGTGTTGTTAGTTGGTCC |
| 117 | AGAGCCACGAGGGCCACGAT |
| 118 | AGAGGCCAGGAGAGCCACGA |
| 119 | CAGCTCGGGCAGAGGCCAGG |
| 120 | TCTCCCTCATCAGCTCGGGC |
| 121 | GCCCAGCAGCAAGGATCCCT |
| 122 | CCTGCATAGAGCCCAGCAGC |
| 123 | TCCATGTAGCCCTGCATAGA |
| 124 | GGACCGTCTTGGAGGCTTGT |
| 125 | AGTGCATCCTGGACCGTCTT |
| 126 | CATGCTGCTTAGTGCATCCT |
| 127 | CAGACTCCTGCATGCTGCTT |
| 128 | ACAGCTATATCAGACTCCTG |
| 129 | CTGGCCACCACAGCTATATC |
| 130 | AAGCGATTGTCCATCCAGCC |
| 131 | TGCTCCAGTAGCCTTTCAGG |
| 132 | GAACTTGCTCCAGTAGCCTT |
| 133 | CAGTGAACTTGCTCCAGTAG |
| 134 | CTTATCAGTGAACTTGCTCC |
| 135 | CCAGTGAACTTATCAGTGAA |
| 136 | GAGGCCAGTGAACTTATCAG |
| 137 | CCAGAGGCCAGTGAACTTAT |
| 138 | GACTCCCAGAGGCCAGTGAA |
| 139 | GGCCAGACTCCCAGAGGCCA |
| 140 | AGTTGGTCCTCAGGGCCAGA |
| 141 | GTTAGTTGGTCCTCAGGGCC |
| 142 | TGTTGTTAGTTGGTCCTCAG |
| 143 | AGAGTTGGTGTTGTTAGTTG |
| 144 | GCTCAAGAGTTGGTGTTGTT |
| 145 | CACGGCTCAAGAGTTGGTGT |
| 146 | GTCTCACGGCTCAAGAGTTG |
| 147 | GAACATGGAGGTCTCACGGC |
| 148 | TCTGGAACATGGAGGTCTCA |
| 149 | CACATCTGGAACATGGAGGT |
| 150 | CAGACACATCTGGAACATGG |
| 151 | TGGCCAGACACATCTGGAAC |
| 152 | AGGATAGATGGCCAGACACA |
| 153 | CAGCAGGATAGATGGCCAGA |
| 154 | GAGGCAGCAGGATAGATGGC |
| 155 | TTCGGAGGCAGCAGGATAGA |
| 156 | AACCTTCGGAGGCAGCAGGA |
| 157 | GAGCAACCTTCGGAGGCAGC |
| 158 | CTTAGAGCAACCTTCGGAGG |
| 159 | TCCCCTTAGAGCAACCTTCG |
| 160 | ACTTTCCCCTTAGAGCAACC |
| 161 | ATATACTTTCCCCTTAGAGC |
| 162 | GAGAATATACTTTCCCCTTA |
| 163 | GCATGAGAATATACTTTCCC |
| 164 | AAAGGCATGAGAATATACTT |
| 165 | GGATAAAGGCATGAGAATAT |
| 166 | GGAGGGATAAAGGCATGAGA |
| 167 | GCATGTTTAGGTGAGGTCTG |
| 168 | GACAGCATGTTTAGGTGAGG |
| 169 | TTATTTGGGACAGCATGTTT |
| 170 | GCTTTTATTTGGGACAGCAT |
| 171 | TCCCAGCTTTTATTTGGGAC |
| 172 | CACGATGAGGAGCATTCGGG |
| 173 | AGGGCCACGATGAGGAGCAT |
| 174 | CCACGAGGGCCACGATGAGG |
| 175 | GAGAGCCACGAGGGCCACGA |
| 176 | GCCAGGAGAGCCACGAGGGC |
| 177 | CAGAGGCCAGGAGAGCCACG |
| 178 | TCGGGCAGAGGCCAGGAGAG |
| 179 | TCAGCTCGGGCAGAGGCCAG |
| 180 | CCTCATCAGCTCGGGCAGAG |
| 181 | CTCTCCCTCATCAGCTCGGG |
| 182 | GATCCCTCTCCCTCATCAGC |
| 183 | GCAAGGATCCCTCTCCCTCA |
| 184 | CAGCAGCAAGGATCCCTCTC |
| 185 | GAGCCCAGCAGCAAGGATCC |
| 186 | GCATAGAGCCCAGCAGCAAG |
| 187 | GCCCTGCATAGAGCCCAGCA |
| 188 | ATGTAGCCCTGCATAGAGCC |
| 189 | GTTCCATGTAGCCCTGCATA |
| 190 | GGCTTGTTCCATGTAGCCCT |
| 191 | TTGGAGGCTTGTTCCATGTA |
| 192 | CCGTCTTGGAGGCTTGTTCC |
| 193 | CTGGACCGTCTTGGAGGCTT |
| 194 | GCATCCTGGACCGTCTTGGA |
| 195 | TTAGTGCATCCTGGACCGTC |
| 196 | GCTGCTTAGTGCATCCTGGA |
| 197 | TGCATGCTGCTTAGTGCATC |
| 198 | ACTCCTGCATGCTGCTTAGT |
| 199 | ATCAGACTCCTGCATGCTGC |
| 200 | GCTATATCAGACTCCTGCAT |
| 201 | CCACAGCTATATCAGACTCC |
| 202 | GGCCACCACAGCTATATCAG |
| 203 | CTGCTGGCCACCACAGCTAT |
| 204 | AGCCCCTGCTGGCCACCACA |
| 205 | CATCCAGCCCCTGCTGGCCA |
| 206 | TTGTCCATCCAGCCCCTGCT |
| 207 | ATTGTCCATCCAGCCCCTGC |
| 208 | AGCGATTGTCCATCCAGCCC |
| 209 | TTTGAAGCGATTGTCCATCC |
| 210 | AGGGATTTGAAGCGATTGTC |
| 211 | CTTTCAGGGATTTGAAGCGA |
| 212 | GTAGCCTTTCAGGGATTTGA |
| 213 | CTCCAGTAGCCTTTCAGGGA |
| 214 | ACTTGCTCCAGTAGCCTTTC |
| 215 | AGTGAACTTGCTCCAGTAGC |
| 216 | TTATCAGTGAACTTGCTCCA |
| 217 | GCCAGTCAACTTATCAGTGA |
| 218 | CAGAGGCCAGTGAACTTATC |
| 219 | ACTCCCAGAGGCCAGTGAAC |
| 220 | GCCAGACTCCCAGAGGCCAG |
| 221 | TAGTTGGTCCTCAGGGCCAG |
| 222 | GTTGTTAGTTGGTCCTCAGG |
| 223 | AAGAGTTGGTGTTGTTAGTT |
| 224 | GGCTCAAGAGTTGGTGTTGT |
| 225 | TCACGGCTCAAGAGTTGGTG |
| 226 | GGAGGCTTGTTCCATGTAGC |
| 227 | TCAGGGATTTGAAGCGATTG |
| 228 | CAGTAGCCTTTCAGGGATTT |
| 229 | ATGGAGGTCTCACGGCTCAA |
| 230 | TAGATGGCCAGACACATCTG |
| 231 | TTGGGACAGCATGTTTAGGT |
| 232 | TGCCAAGAGGGCAACAATAG |
| 233 | AGGAGTGCCAAGAGGGCAAC |
| 234 | GATGCCAGGAGTGCCAAGAG |
| 235 | GGCAGATGCCAGGAGTGCCA |
| 236 | CTCTACCTCATTAGCTTCGG |
| 237 | CCCTCTACCTCATTAGCTTC |
| 238 | GACCCCTCTACCTCATTAGC |
| 239 | GCAAGGACCCCTCTACCTCA |
| 240 | CAGCAGCAAGGACCCCTCTA |
| 241 | GAGCCCAGCAGCAAGGACCC |
| 242 | TGCACAGAGCCCAGCAGCAA |
| 243 | AGCCCTGCACAGAGCCCAGC |
| 244 | CATGTAGCCCTGCACAGAGC |
| 245 | TGTTCCATGTAGCCCTGCAC |
| 246 | TGGCCTGTTCCATGTAGCCC |
| 247 | ACCTTCTTGGTGGCCTGTTC |
| 248 | GCGCATCCTGGACCTTCTTG |
| 249 | TGCTGGTTAGCGCATCCTGG |
| 250 | TTGCATGCTGGTTAGCGCAT |
| 251 | GACTTTTGCATGCTGGTTAG |
| 252 | CCTCAGACTTTTGCATGCTG |
| 253 | AGCCACCTCAGACTTTTGCA |
| 254 | CGCACAGCCACCTCAGACTT |
| 255 | CCAGTCCCTGGCCCGCACAG |
| 256 | GTCCATCCAGTCCCTGGCCC |
| 257 | CCGTCCATCCAGTCCCTGGC |
| 258 | GCCACCGTCCATCCAGTCCC |
| 259 | GTGAAGCCACCGTCCATCCA |
| 260 | GGAGGTGAAGCCACCGTCCA |
| 261 | TGCTCCAGTAGCTTTTCAGG |
| 262 | GTAAATGTGCTCCAGTAGCT |
| 263 | TGTCAGTAAATGTGCTCCAG |
| 264 | TGGAGACCGTGTCAGTAAAT |
| 265 | GGCTGGAGACCGTGTCAGTA |
| 266 | CAGAGGCTGGAGACCGTGTC |
| 267 | ATCCCAGAGGCTGGAGACCG |
| 268 | GAAGAATCCCAGAGGCTGGA |
| 269 | TCTCAAGGCTCAGTAGCTGG |
| 270 | TAGAGGTCTCAAGGCTCAGT |
| 271 | GAACGTAGAGGTCTCAAGGC |
| 272 | CATTTGGAACGTAGAGGTCT |
| 273 | CAAGCACATTTGGAACGTAG |
| 274 | TGGACACACAAGCACATTTG |
| 275 | CAGGATGGACACACAAGCAC |
| 276 | GGCCAGCAGGATGGACACAC |
| 277 | GCCCAGAGGCCAGCAGGATG |
| 278 | CCTTTCAAACAACCTTCAGG |
| 279 | GGACAGCATGTTTAGGTGAC |
| 280 | CTTGGTGGCCTGTTCCATGT |
| 281 | GTTAGCGCATCCTGGACCTT |
| 282 | TGGCCCGCACAGCCACCTCA |
| 283 | GACCGTGTCAGTAAATGTGC |
| 284 | AAGAGGGCAACAATAGGAGT |
| 285 | GTGCCAAGAGGGCAACAATA |
| 286 | ATGCCAGGAGTGCCAAGAGG |
| 287 | CTTCGGGCAGATGCCAGGAG |
| 288 | CATTAGCTTCGGGCAGATGC |
| 289 | AGAGCCCAGCAGCAAGGACC |
| 290 | GTGGCCTGTTCCATGTAGCC |
| 291 | TCTTGGTGGCCTGTTCCATG |
| 292 | GACCTTCTTGGTGGCCTGTT |
| 293 | TCCTGGACCTTCTTGGTGGC |
| 294 | GGTTAGCGCATCCTGGACCT |
| 295 | ATGCTGGTTAGCGCATCCTG |
| 296 | TTTGCATGCTGGTTAGCGCA |
| 297 | AGACTTTTGCATGCTGGTTA |
| 298 | ACCTCAGACTTTTGCATGCT |
| 299 | CAGCCACCTCAGACTTTTGC |
| 300 | CCGCACAGCCACCTCAGACT |
| 301 | CTGGCCCGCACAGCCACCTC |
| 302 | AGTCCCTGGCCCGCACAGCC |
| 303 | CATCCAGTCCCTGGCCCGCA |
| 304 | AGCCACCGTCCATCCAGTCC |
| 305 | GGTGAAGCCACCGTCCATCC |
| 306 | AGGGAGGTGAAGCCACCGTC |
| 307 | TTTTCAGGGAGGTGAAGCCA |
| 308 | AGTAGCTTTTCAGGGAGGTG |
| 309 | AAATGTGCTCCAGTAGCTTT |
| 310 | TCAGTAAATGTGCTCCAGTA |
| 311 | CCGTGTCAGTAAATGTGCTC |
| 312 | GGAGACCGTGTCAGTAAATG |
| 313 | AGGCTGGAGACCGTGTCAGT |
| 314 | CCCAGAGGCTGGAGACCGTG |
| 315 | AGAATCCCAGAGGCTGGAGA |
| 316 | AGAGGTCTCAAGGCTCAGTA |
| 317 | AACGTAGAGGTCTCAAGGCT |
| 318 | TTTGGAACGTAGAGGTCTCA |
| 319 | GCACATTTGGAACGTAGAGG |
| 320 | CACAAGCACATTTGGAACGT |
| 321 | GGACACACAAGCACATTTGG |
| 322 | AGGATGGACACACAAGCACA |
| 323 | CCAGCAGGATGGACACACAA |
| 324 | AGAGGCCAGCAGGATGGAGA |
| 325 | GGCCCAGAGGCCAGCAGGAT |
| 326 | ACCCAGGCCCAGAGGCCAGC |
| 327 | GGGCCACCCAGGCCCAGAGG |
| 328 | CTTTCCCTTTCAAACAACCT |
| 329 | CAATACTTTCCCTTTCAAAC |
| 330 | CATGACAATACTTTCCCTTT |
| 331 | GAAAACATGACAATACTTTC |
| 332 | GGGATGAAAACATGACAATA |
| 333 | CATGTTTAGGTGACTTCTGG |
| 334 | GACAGCATGTTTAGGTGACT |
| 335 | TTTAGGACAGCATGTTTAGG |
| 336 | CTTTATTTAGGACAGCATGT |
| 337 | TCCAGCTTTATTTAGGACAG |

In some embodiments, the Apo-C3 modulating compound comprises the nucleobase sequence of SEQ ID NO.:69. In such embodiments, the method of reducing triglycerides in a subject on Apo-C3 modulating therapy comprises administering to the subject a pharmaceutical composition comprising at least about 80%, by weight of all fatty acids and/or derivatives thereof (e.g., at least 80%, at least about 90%, at least about 95%, or at least about 96%, by weight of all fatty acids and/or derivatives thereof), ethyl eicosapentaenoate. Alternatively, in other embodiments the method comprises administering to the subject on Apo-C3 modulating therapy about 1 to about 4 capsules per day (e.g., 1 capsule, 2 capsules, 3 capsules or 4 capsules per day), each capsule comprising about 1 g of ethyl eicosapentaenoate.

In some embodiments, the Apo-C3 modulating compound (including any of those disclosed in Table 1) is a chimeric oligonucleotide (e.g., a “gapmer”) comprising, consisting essentially of, or consisting a central “gap” region flanked on both sides (i.e., on both the 5′ and the 3′ sides) by nucleotide “wings.” In some embodiments, the central gap region includes 5, 6, 7, 8, 9, 10, 11, 12, 13 or 14 nucleotides. In some embodiments, the nucleotides in the central gap region are modified nucleotides, such as 2′-deoxynucleotides. In some embodiments, the nucleotide wings each individually include 1, 2, 3, 4, 5, 6, 7, 8, 9 or 10 nucleotides. In some embodiments, the nucleotide wings individually comprise, consist essentially of, or consist of modified nucleotides, such as 2′-O-(2-methoxyethyl) nucleotides (also referred to as “2′-MOE nucleotides”). In some embodiments, one or more (or all) cytidine residues in an Apo-C3 modulating compound of the present disclosure are 5-methylcytidine residues. In some embodiments, one or more (or all) internucleoside (e.g., backbone) linkages are phosphorothioate (P═S) linkages. In some embodiments, the Apo-C3 modulating compound is in a pharmaceutically acceptable salt form. In some embodiments, the Apo-C3 modulating compound is capable of inhibiting human Apo-C3 expression in an in vitro assay by at least 30%, at least 35%, at least 40%, at least 45%, at least 50%, at least 55%, at least 60%, at least 65%, at least 70%, at least 74%, at least 75%, at least 80%, at least 85%, at least 90%, at least 95%, at least 99%, or more than 99%. Suitable in vitro assays are well-known in the art.

Co-administration of an Apo-C3 modulating compound and eicosapentaenoic acid (or a derivative thereof) can be accomplished by any suitable means. For example, and without limitation, the Apo-C3 modulating compound can be admixed, encapsulated, conjugated or otherwise associated with the eicosapentaenoic acid (or derivative thereof). In some embodiments, the Apo-C3 modulating compound and the eicosapentaenoic acid (or derivative thereof) are co-administered in an oral dosage form, such as by oral capsule (e.g., a gelatin capsule). Alternatively, the Apo-C3 modulating compound may be co-administered with the eicosapentaenoic acid (or derivative thereof) in separate dosage forms. For example, in one such embodiment the eicosapentaenoic acid (or derivative thereof) is orally administered (e.g., in oral capsule or gelatin capsule form), while the Apo-C3 modulating compound is administered topically (e.g., to ophthalmic or mucous membranes), pulmonarily (e.g., by inhalation or insufflation of powders or aerosols), or parenterally (e.g., intravenously, intraarterially, subcutaneously, by intraperitoneal or intramuscular injection or infusion, or intracranially).

In one embodiment, co-administration of eicosapentaenoic acid (or a derivative thereof) and an Apo-C3 modulating compound comprises administering the Apo-C3 modulating compound to a subject in an amount of about 0.01 μg to about 100 g per kilogram of body weight (about 0.01 μg/kg to about 100 g/kg). In some embodiments, the Apo-C3 modulating compound is administered to the subject once or more daily (e.g., one time per day, 2 times per day, 3 times per day, 4 times per day, 5 times per day, 6 times per day, 7 times per day, 8 times per day, 9 times per day, 10 times per day, or more than 10 times per day) to once every 20 years.

In some embodiments, a method of reducing triglycerides in a subject on Apo-C3 modulating therapy according to the present invention comprises administering to the subject a composition comprising at least about 80%, by weight of all fatty acids (and/or derivatives thereof) present, ethyl eicosapentaenoate. In other embodiments, a method of reducing triglycerides in a subject on Apo-C3 modulating therapy according to the present invention comprises administering to the subject about 1 to about 4 capsules per day, each capsule comprising about 1 g of ethyl eicosapentaenoate. In some embodiments, the capsules comprise at least about 80%, by weight of all fatty acids (and/or derivatives thereof) present, ethyl eicosapentaenoate.

In some embodiments, the subject has a fasting baseline triglyceride level of about 200 mg/dl to 499 mg/dl. In some embodiments, the second subject or second subject group has a fasting baseline triglyceride level or a mean or median fasting baseline triglyceride level of about 200 mg/dl to 499 mg/dl. In some embodiments, the subject has a fasting baseline triglyceride level of at least 500 mg/dl. In some embodiments, the second subject or second subject group has a fasting baseline triglyceride level or a mean or median fasting baseline triglyceride level of at least 500 mg/dl. In some embodiments, triglycerides are reduced in the subject with no increase in an LDL-C level in the subject. In some embodiments, the reduction in triglycerides and the no increase in LDL-C level is in comparison to baseline or to a second subject or subject group that has received an Apo-C3 modulating compound but not the ethyl eicosapentaenoate.

A composition for use in accordance with the invention can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In another embodiment, the invention provides use of any composition described herein for treating moderate to severe hypertriglyceridemia in a subject in need thereof, comprising: providing a subject having a fasting baseline triglyceride level of 500 mg/dl to about 1500 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid. In some embodiments, cholesterol domain formation in membranes of the subject is reduced or prevented. In some embodiments, the subject experiences no substantial increase, or no increase, or a reduction, in LDL-C levels. In some embodiments, the subject is on Apo-C3 modulating therapy.

In another embodiment, the invention provides use of any composition described herein for treating moderate to severe hypertriglyceridemia in a subject in need thereof, comprising: providing a subject on statin therapy and having a fasting baseline triglyceride level of about 200 mg/dl to 499 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid. In some embodiments, cholesterol domain formation in membranes of the subject is reduced or prevented. In some embodiments, the subject experiences no substantial increase, or no increase, or a reduction, in LDL-C levels. In some embodiments, the subject is on Apo-C3 modulating therapy.

In one embodiment, compositions of the invention, upon storage in a closed container maintained at room temperature, refrigerated (e.g. about 5 to about 5-10° C.) temperature, or frozen for a period of about 1, 2, 3, 4, 5, 6, 7, 8, 9, 10, 11, or 12 months, exhibit at least about 90%, at least about 95%, at least about 97.5%, or at least about 99% of the active ingredient(s) originally present therein.

In one embodiment, the invention provides use of a composition as described herein in manufacture of a medicament for treatment of any of a cardiovascular-related disease. In another embodiment, the subject is diabetic.

In one embodiment, a composition as set forth herein is packaged together with instructions for using the composition to treat a cardiovascular disorder.

### EXAMPLES

### Example 1

A multi-center, placebo-controlled randomized, double-blind, 12-week study with an open-label extension was performed to evaluate the efficacy and safety of AMR101 in patients with fasting triglyceride levels ≧500 mg/dL. The primary objective of the study was to determine the efficacy of AMR101 2 g daily and 4 g daily, compared to placebo, in lowering fasting TG levels in patients with fasting TG levels ≧500 mg/dL and ≦1500 mg/dL (≧5.65 mmol/L and ≦16.94 mmol/L).

The secondary objectives of this study were the following:
To determine the safety and tolerability of AMR101 2 g daily and 4 g daily;
To determine the effect of AMR101 on lipid and apolipoprotein profiles;
To determine the effect of AMR101 on low-density lipoprotein (LDL) particle number and size;
To determine the effect of AMR101 on oxidized LDL;
To determine the effect of AMR101 on fasting plasma glucose (FPG) and hemoglobin A1c (HbA1c);
To determine the effect of AMR101 on insulin resistance;
To determine the effect of AMR101 on high-sensitivity C-reactive protein (hsCRP);
To determine the effects of AMR101 2 g daily and 4 g daily on the incorporation of fatty acids into red blood cell membranes and into plasma phospholipids;
To explore the relationship between baseline fasting TG levels and the reduction in fasting TG levels; and
To explore the relationship between an increase in red blood cell membrane eicosapentaenoic acid (EPA) concentrations and the reduction in fasting TG levels.

The population for this study was men and women (women of childbearing potential needed to be on contraception or practice abstinence)>18 years of age with a body mass index ≦45 kg/m² who were not on lipid altering therapy or were not currently on lipid altering therapy. Patients currently on statin therapy (with or without ezetimibe) were evaluated by the investigator as to whether this therapy could be safely discontinued at screening, or if it should have been continued. If statin therapy (with or without ezetimibe) was to be continued, dose(s) must have been stable for ≧4 weeks prior to randomization. Patients taking non-statin, lipid-altering medications (niacin >200 mg/day, fibrates, fish oil, other products containing omega 3 fatty acids, or other herbal products or dietary supplements with potential lipid altering effects), either alone or in combination with statin therapy (with or without ezetimibe), must have been able to safely discontinue non-statin, lipid altering therapy at screening.

Approximately 240 patients were randomized at approximately 50 centers in North America, South America, Central America, Europe, India, and South Africa. The study was a 58- to 60-week, Phase 3, multi-center study consisting of 3 study periods: (1) a 6- to 8-week screening period that included a diet and lifestyle stabilization and washout period and a TG qualifying period; (2) a 12-week, double-blind, randomized, placebo controlled treatment period; and (3) a 40-week, open-label, extension period.

During the screening period and double-blind treatment period, all visits were within ±3 days of the scheduled time. During the open-label extension period, all visits were within ±7 days of the scheduled time. The screening period included a 4- or 6-week diet and lifestyle stabilization period and washout period followed by a 2-week TG qualifying period.

The screening visit (Visit 1) occurred for all patients at either 6 weeks (for patients not on lipid-altering therapy at screening or for patients who did not need to discontinue their current lipid-altering therapy) or 8 weeks (for patients who required washout of their current lipid altering therapy at screening) before randomization, as follows:

Patients who did not require a washout: The screening visit will occur at Visit 1 (Week −6). Eligible patients entered a 4 week diet and lifestyle stabilization period. At the screening visit, all patients received counseling regarding the importance of the National Cholesterol Education Program (NCEP) Therapeutic Lifestyle Changes (TLC) diet and received instructions on how to follow this diet. Patients who required a washout: The screening visit occurred at Visit 1 (Week −8). Eligible patients began a 6 week washout period at the screening visit. Patients received counseling regarding the NCEP TLC diet and received instructions on how to follow this diet. Site personnel contacted patients who did not qualify for participation based on screening laboratory test results to instruct them to resume their prior lipid altering medications.

At the end of the 4-week diet and lifestyle stabilization period or the 6-week diet and stabilization and washout period, eligible patients entered the 2-week TG qualifying period and had their fasting TG level measured at Visit 2 (Week −2) and Visit 3 (Week −1). Eligible patients must have had an average fasting TG level ≧500 mg/dL and ≦1500 mg/dL (≧5.65 mmol/L and ≦16.94 mmol/L) to enter the 12 week double blind treatment period. The TG level for qualification was based on the average (arithmetic mean) of the Visit 2 (Week −2) and Visit 3 (Week −1) values. If a patient's average TG level from Visit 2 and Visit 3 fell outside the required range for entry into the study, an additional sample for fasting TG measurement was collected 1 week later at Visit 3.1. If a third sample was collected at Visit 3.1, entry into the study was based on the average (arithmetic mean) of the values from Visit 3 and Visit 3.1.

After confirmation of qualifying fasting TG values, eligible patients entered a 12-week, randomized, double-blind treatment period. At Visit 4 (Week 0), patients were randomly assigned to one of the following treatment groups:
AMR101 2 g daily,
AMR101 4 g daily, or
Placebo.

During the double-blind treatment period, patients returned to the site at Visit 5 (Week 4), Visit 6 (Week 11), and Visit 7 (Week 12) for efficacy and safety evaluations.

Patients who completed the 12-week double-blind treatment period were eligible to enter a 40 week, open label, extension period at Visit 7 (Week 12). All patients received open label AMR101 4 g daily. From Visit 8 (Week 16) until the end of the study, changes to the lipid-altering regimen were permitted (e.g., initiating or raising the dose of statin or adding non-statin, lipid-altering medications to the regimen), as guided by standard practice and prescribing information. After Visit 8 (Week 16), patients returned to the site every 12 weeks until the last visit at Visit 11 (Week 52).

Eligible patients were randomly assigned at Visit 4 (Week 0) to orally receive AMR101 2 g daily, AMR101 4 g daily, or placebo for the 12-week double-blind treatment period. AMR101 was provided in 1 g liquid-filled, oblong, gelatin capsules. The matching placebo capsule was filled with light liquid paraffin and contained 0 g of AMR101. During the double-blind treatment period, patients took 2 capsules (AMR101 or matching placebo) in the morning and 2 in the evening for a total of 4 capsules per day. Patients in the AMR101 2 g/day treatment group received 1 AMR101 1 g capsule and 1 matching placebo capsule in the morning and in the evening. Patients in the AMR101 4 g/day treatment group received 2 AMR101 1 g capsules in the morning and evening.

Patients in the placebo group received 2 matching placebo capsules in the morning and evening. During the extension period, patients received open-label AMR101 4 g daily. Patients took 2 AMR101 1 g capsules in the morning and 2 in the evening.

The primary efficacy variable for the double-blind treatment period was percent change in TG from baseline to Week 12 endpoint. The secondary efficacy variables for the double-blind treatment period included the following:
Percent changes in total cholesterol (TC), high-density lipoprotein cholesterol (HDL-C), calculated low density lipoprotein cholesterol (LDL-C), calculated non-high-density lipoprotein cholesterol (non-HDL-C), and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A I/apo B ratio from baseline to Week 12;
Percent changes in lipoprotein(a) from baseline to Week 12 (selected sites only);
Percent changes in LDL particle number and size, measured by nuclear magnetic resonance, from baseline to Week 12 (selected sites only);
Percent change in remnant-like particle cholesterol from baseline to Week 12 (selected sites only);
Percent change in oxidized LDL from baseline to Week 12 (selected sites only);
Changes in FPG and HbA1c from baseline to Week 12;
Change in insulin resistance, as assessed by the homeostasis model index insulin resistance, from baseline to Week 12;
Percent change in lipoprotein associated phospholipase A2 from baseline to Week 12 (selected sites only);
Change in intracellular adhesion molecule-1 from baseline to Week 12 (selected sites only);
Change in interleukin-6 from baseline to Week 12 (selected sites only);
Change in plasminogen activator inhibitor-1 from baseline to Week 12 (selected sites only);
Change in hsCRP from baseline to Week 12 (selected sites only);
Change in serum phospholipid EPA content from baseline to Week 12;
Change in red blood cell membrane EPA content from baseline to Week 12; and
Change in serum phospholipid and red blood cell membrane content in the following fatty acids from baseline to Week 12: docosapentaenoic acid, docosahexaenoic acid, arachidonic acid, palmitic acid, stearic acid, and oleic acid.

The efficacy variable for the open-label extension period was percent change in fasting TG from extension baseline to end of treatment. Safety assessments included adverse events, clinical laboratory measurements (chemistry, hematology, and urinalysis), 12-lead electrocardiograms (ECGs), vital signs, and physical examinations

For TG, TC, HDL-C, calculated LDL-C, calculated non-HDL-C, and VLDL-C, baseline was defined as the average of Visit 4 (Week 0) and the preceding lipid qualifying visit (either Visit 3 [Week −1] or if it occurs, Visit 3.1) measurements. Baseline for all other efficacy parameters was the Visit 4 (Week 0) measurement.

For TC, HDL-C, calculated LDL-C, calculated non-HDL-C, and VLDL-C, Week 12 endpoint was defined as the average of Visit 6 (Week 11) and Visit 7 (Week 12) measurements. Week 12 endpoint for all other efficacy parameters was the Visit 7 (Week 12) measurement.

The primary efficacy analysis was performed using a 2-way analysis of covariance (ANCOVA) model with treatment as a factor and baseline TG value as a covariate. The least squares mean, standard error, and 2-tailed 95% confidence interval for each treatment group and for each comparison was estimated. The same 2-way ANCOVA model was used for the analysis of secondary efficacy variables.

The primary analysis was repeated for the per-protocol population to confirm the robustness of the results for the intent-to-treat population.

The primary efficacy variable was the percent change in fasting TG levels from baseline to Week 12. A sample size of 69 completed patients per treatment group was expected to provide ≧90% power to detect a difference of 30% between AMR101 and placebo in percent change from baseline in fasting TG levels, assuming a standard deviation of 45% in TG measurements and a significance level of p<0.01. To accommodate a 15% drop-out rate from randomization to completion of the double-blind treatment period, a total of 240 randomized patients was planned (80 patients per treatment group).

### Example 2

A multi-center, placebo-controlled, randomized, double-blind, 12-week study was performed to evaluate the efficacy and safety of >96% E-EPA in patients with fasting triglyceride levels ≧200 mg/dl and <500 mg/dl despite statin therapy (the mean of two qualifying entry values needed to be ≧185 mg/dl and at least one of the values needed to be ≧200 mg/dl). The primary objective of the study was to determine the efficacy of >96% E-EPA 2 g daily and 4 g daily, compared to placebo, in lowering fasting TG levels in patients with high risk for cardiovascular disease and with fasting TG levels 200 mg/dl and <500 mg/dl, despite treatment to LDL-C goal on statin therapy.

The secondary objectives of this study were the following:
To determine the safety and tolerability of >96% E-EPA 2 g daily and 4 g daily;
To determine the effect of >96% E-EPA on lipid and apolipoprotein profiles including total cholesterol (TC), non-high-density lipoprotein cholesterol (non-HDL-C), low density lipoprotein cholesterol (LDL-C), high density lipoprotein cholesterol (HDL-C), and very high density lipoprotein cholesterol (VHDL-C);
To determine the effect of >96% E-EPA on lipoprotein associated phospholipase A2 (Lp-PLA2) from baseline to week 12;
To determine the effect of >96% E-EPA on low-density lipoprotein (LDL) particle number and size;
To determine the effect of >96% E-EPA on oxidized LDL;
To determine the effect of >96% E-EPA on fasting plasma glucose (FPG) and hemoglobin A1c (HbA1c);
To determine the effect of >96% E-EPA on insulin resistance;
To determine the effect of >96% E-EPA on high-sensitivity C-reactive protein (hsCRP);
To determine the effects of >96% E-EPA 2 g daily and 4 g daily on the incorporation of fatty acids into red blood cell membranes and into plasma phospholipids;
To explore the relationship between baseline fasting TG levels and the reduction in fasting TG levels; and
To explore the relationship between changes of fatty acid concentrations in plasma and red blood cell membranes, and the reduction in fasting TG levels.

The population for this study was men and women >18 years of age with a body mass index <45 kg/m² with fasting TG levels greater than or equal to 200 mg/dl and less than 500 mg/dl and on a stable does of statin therapy (with or without ezetimibe). The statin was atorvostatin, rosuvastatin or simvastatin. The dose of statin must have been stable for ≧4 weeks prior to the LDL-C/TG baseline qualifying measurement for randomization. The statin dose was optimized such that the patients are at their LDL-C goal at the LDL-C/TG baseline qualifying measurements. The same statin at the same dose was continued until the study ended.

Patients taking any additional non-statin, lipid-altering medications (niacin >200 mg/day, fibrates, fish oil, other products containing omega 3 fatty acids, or other herbal products or dietary supplements with potential lipid altering effects), either alone or in combination with statin therapy (with or without ezetimibe), must have been able to safely discontinue non-statin, lipid altering therapy at screening. Patients at high risk for CVD, i.e., patients with clinical coronary heart disease (CHD) or clinical CHD risk equivalents (10-year risk >20%) as defined in the National Cholesterol Education Program (NCEP) Adult Treatment Panel III (ATP III) Guidelines were eligible to participate in this study. Those included patients with any of the following criteria: (1) Known CVD, either clinical coronary heart disease (CHD), symptomatic carotid artery disease (CAD), peripheral artery disease (PAD) or abdominal aortic aneurism; or (2) Diabetes Mellitus (Type 1 or 2).

Approximately 702 patients were randomized at approximately 80 centers in the U.S. The study was a 18- to 20-week, Phase 3, multi-center study consisting of 2 study periods: (1) A 6- to 8-week screening period that included a diet and lifestyle stabilization, a non-statin lipid-altering treatment washout, and an LDL-C and TG qualifying period and (2) A 12-week, double-blind, randomized, placebo controlled treatment period.

During the screening period and double-blind treatment period, all visits were within ±3 days of the scheduled time. All patients continued to take the statin product (with or without ezetimibe) at the same dose they were taking at screening throughout their participation in the study.

The 6- to 8-week screening period included a diet and lifestyle stabilization, a non-statin lipid-altering treatment washout, and an LDL-C and TG qualifying period. The screening visit (Visit 1) occurred for all patients at either 6 weeks (for patients on stable statin therapy [with or without ezetimibe] at screening) or 8 weeks (for patients who will require washout of their current non-statin lipid altering therapy at screening) before randomization, as follows:
Patients who did not require a washout: The screening visit occurred at Visit 1 (Week −6). Eligible patients entered a 4 week diet and lifestyle stabilization period. At the screening visit, all patients received counseling regarding the importance of the National Cholesterol Education Program (NCEP) Therapeutic Lifestyle Changes (TLC) diet and received basic instructions on how to follow this diet.
Patients who required a washout: The screening visit occurred at Visit 1 (Week −8). Eligible patients began a 6 week washout period at the screening visit (i.e. 6 weeks washout before the first LDL-C/TG qualifying visit). Patients received counseling regarding the NCEP TLC diet and received basic instructions on how to follow this diet. Site personnel contacted patients who did not qualify for participation based on screening laboratory test results to instruct them to resume their prior lipid altering medications.

At the end of the 4-week diet and lifestyle stabilization period or the 6-week diet and stabilization and washout period, eligible patients entered the 2-week LDL-C and TG qualifying period and had their fasting LDL-C and TG levels measured at Visit 2 (Week 2) and Visit 3 (Week −1). Eligible patients must have had an average fasting LDL-C level >40 mg/dL and <100 mg/dL and an average fasting TG level >200 mg/dL and <500 mg/dL to enter the 12 week double blind treatment period. The LDL-C and TG levels for qualification were based on the average (arithmetic mean) of the Visit 2 (Week −2) and Visit 3 (Week −1) values. If a patient's average LDL-C and/or TG levels from Visit 2 and Visit 3 fell outside the required range for entry into the study, an additional fasting lipid profile was collected 1 week later at Visit 3.1. If a third sample was collected at Visit 3.1, entry into the study was based on the average (arithmetic mean) of the values from Visit 3 and Visit 3.1.

After confirmation of qualifying fasting LDL-C and TG values, eligible patients entered a 12-week, randomized, double-blind treatment period. At Visit 4 (Week 0), patients were randomly assigned to 1 of the following treatment groups:
>96% E-EPA 2 g daily,
>96% E-EPA 4 g daily, or
Placebo.

226 to 234 patients per treatment group were randomized in this study. Stratification was by type of statin (atorvastatin, rosuvastatin or simvastatin), the presence of diabetes, and gender.

During the double-blind treatment period, patients returned to the site at Visit 5 (Week 4), Visit 6 (Week 11), and Visit 7 (Week 12) for efficacy and safety evaluations.

Eligible patients were randomly assigned at Visit 4 (Week 0) to receive orally >96% E-EPA 2 g daily, >96% E-EPA 4 g daily, or placebo.

>96% E-EPA was provided in 1 g liquid-filled, oblong, gelatin capsules. The matching placebo capsule was filled with light liquid paraffin and contained 0 g of >96% E-EPA. >96% E-EPA capsules were to be taken with food (i.e. with or at the end of a meal).

During the double-blind treatment period, patients were to take 2 capsules (>96% E-EPA or matching placebo) in the morning and 2 capsules in the evening for a total of 4 capsules per day.
Patients in the >96% E-EPA 2 g/day treatment group received 1 >96% E-EPA 1 g capsule and 1 matching placebo capsule in the morning and in the evening.
Patients in the >96% E-EPA 4 g/day treatment group received 2 >96% E-EPA 1 g capsules in the morning and evening.
Patients in the placebo group received 2 matching placebo capsules in the morning and evening.

The primary efficacy variable for the double-blind treatment period was percent change in TG from baseline to Week 12 endpoint. The secondary efficacy variables for the double-blind treatment period included the following:
Percent changes in total cholesterol (TC), high-density lipoprotein cholesterol (HDL-C), LDL-C, calculated non-HDL-C, and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A I/apo B ratio from baseline to Week 12;
Percent changes in lipoprotein(a) from baseline to Week 12;
Percent changes in LDL particle number and size, measured by nuclear magnetic resonance, from baseline to Week 12;
Percent change in remnant-like particle cholesterol from baseline to Week 12;
Percent change in oxidized LDL from baseline to Week 12;
Changes in FPG and HbA1c from baseline to Week 12;
Change in insulin resistance, as assessed by the homeostasis model index insulin resistance, from baseline to Week 12;
Percent change in lipoprotein associated phospholipase A2 (Lp-PLA2) from baseline to Week 12;
Change in intracellular adhesion molecule-1 from baseline to Week 12;
Change in interleukin-2 from baseline to Week 12;
Change in plasminogen activator inhibitor-1 from baseline to Week 12. Note: this parameter will only be collected at sites with proper storage conditions;
Change in hsCRP from baseline to Week 12; and
Change in plasma concentration and red blood cell membrane content of fatty acid from baseline to Week 12 including EPA, docosapentaenoic acid (DPA), docosahexaenoic acid (DHA), arachidonic acid (AA), dihomo-γ-linolenic acid (DGLA), the ratio of EPA/AA, ratio of oleic acid/stearic acid (OA/SA), and the ratio of total omega-3 acids over total omega-6 acids.

Safety assessments included adverse events, clinical laboratory measurements (chemistry, hematology, and urinalysis), 12-lead electrocardiograms (ECGs), vital signs, and physical examinations.

For TG, TC, HDL-C, LDL-C, calculated non-HDL-C, and VLDL-C, baseline was defined as the average of Visit 4 (Week 0) and the preceding lipid qualifying visit (either Visit 3 [Week −1] or if it occurs, Visit 3.1) measurements. Baseline for all other efficacy parameters was the Visit 4 (Week 0) measurement.

For TG, TC, HDL-C, LDL-C, calculated non-HDL-C, and VLDL-C, Week 12 endpoint was defined as the average of Visit 6 (Week 11) and Visit 7 (Week 12) measurements.

Week 12 endpoint for all other efficacy parameters were the Visit 7 (Week 12) measurement.

The primary efficacy analysis was performed using a 2-way analysis of covariance (ANCOVA) model with treatment as a factor and baseline TG value as a covariate. The least squares mean, standard error, and 2-tailed 95% confidence interval for each treatment group and for each comparison were estimated. The same 2-way ANCOVA model was used for the analysis of secondary efficacy variables.

The primary analysis was repeated for the per-protocol population to confirm the robustness of the results for the intent-to-treat population.

Non-inferiority tests for percent change from baseline in LDL-C were performed between >96% E-EPA doses and placebo using a non-inferiority margin of 6% and a significant level at 0.05.

For the following key secondary efficacy parameters, treatment groups were compared using Dunnett's test to control the Type 1 error rate: TC, LDL-C, HDL-C, non-HDL-C, VLDL-C, Lp-PLA2, and apo B. For the remaining secondary efficacy parameters, Dunnett's test was be used and the ANCOVA output were considered descriptive.

The evaluation of safety was based primarily on the frequency of adverse events, clinical laboratory assessments, vital signs, and 12-lead ECGs. The primary efficacy variable is the percent change in fasting TG levels from baseline to Week 12. A sample size of 194 completed patients per treatment group provided 90.6% power to detect a difference of 15% between >96% E-EPA and placebo in percent change from baseline in fasting TG levels, assuming a standard deviation of 45% in TG measurements and a significance level of p<0.05.

Previous data on fasting LDL-C show a difference in percent change from baseline of 2.2%, with a standard deviation of 15%, between study drug and placebo. A sample size of 194 completed patients per treatment group provided 80% power to demonstrate non inferiority (p<0.05, one-sided) of the LDL-C response between >96% E-EPA 4 g daily and placebo, within a 6% margin. To accommodate a 10% drop-out rate from randomization to completion of the double-blind treatment period, a total of 648 randomized patients was planned (216 patients per treatment group); 702 subjects were randomized, as further described below.

### Results

Of the 702 randomized subjects, 687 were in the intent-to-treat (“ITT”) population as follows:
Ultra-pure EPA, 4 g/day: 226 subjects
Ultra-pure EPA, 2 g/day: 234 subjects
Placebo: 227 subjects

Lipids were extracted from plasma and red blood cell (“RBC”) suspensions and converted into fatty acid methyl esters for analysis using a standard validated gas chromatography/flame ionization detection method. Fatty acid parameters were compared between EPA treatment groups and placebo using an ANCOVA model with treatment, gender, type of statin therapy, and presence of diabetes as factors, and the baseline parameter value as a covariate. LSMs, SEs, and 2-tailed 95% confidence intervals for each treatment group and for each comparison were determined.

Baseline characteristics of the three ITT groups were comparable, with 61.4% of the ITT subjects being male, 96.3% being white, having a mean age of 61.4 years, a weight of 95.7 kg and a BMI of 32.9 kg/m². ITT subjects with incomplete fatty acid data at baseline and/or at 12 weeks were excluded from the analyses described below.

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A method of reducing triglycerides in a subject on Apo-C3 modulating therapy, the method comprising administering to the subject a pharmaceutical composition comprising at least about 80%, by weight of all fatty acids (and/or derivatives thereof) present, ethyl eicosapentaenoate.

2. The method of claim 1, wherein the subject has a fasting baseline triglyceride level of about 200 mg/dl to 499 mg/dl.

3. The method of claim 1, wherein the subject has a fasting baseline triglyceride level of at least 500 mg/dl.

4. The method of claim 1, wherein triglycerides are reduced in the subject with no increase in an LDL-C level in the subject.

5. The method of claim 4, wherein the reduction in triglycerides and the no increase in LDL-C level is in comparison to baseline or to a second subject or subject group that has received Apo-C3 modulating therapy but not the ethyl eicosapentaenoate.

7. The method of claim 1, wherein ethyl eicosapentaenoate represents at least about 90%, by weight of all fatty acids (and/or derivatives thereof) present.

8. The method of claim 7, wherein ethyl eicosapentaenoate represents at least about 95%, by weight of all fatty acids (and/or derivatives thereof) present.

9. The method of claim 8, wherein ethyl eicosapentaenoate represents at least about 96%, by weight of all fatty acids (and/or derivatives thereof) present.

10. The method of claim 1, wherein docosahexaenoic acid and its esters represent no more than about 20%, by weight of all fatty acids (and/or derivatives thereof) present in the pharmaceutical composition.

11. The method of claim 10, wherein docosahexaenoic acid and its esters represent no more than about 10%, by weight of all fatty acids (and/or derivatives thereof) present in the pharmaceutical composition.

12. The method of claim 11, wherein docosahexaenoic acid and its esters represent no more than about 5%, by weight of all fatty acids (and/or derivatives thereof) present in the pharmaceutical composition.

13. The method of claim 12, wherein docosahexaenoic acid and its esters represent no more than about 3%, by weight of all fatty acids (and/or derivatives thereof) present in the pharmaceutical composition.

14. The method of claim 1, wherein the Apo-C3 modulating therapy comprises administering to the subject an Apo-C3 modulating compound comprising the nucleobase sequence of any one of SEQ ID NOs: 1-337.

15. The method of claim 14, wherein the Apo-C3 modulating compound comprises the nucleobase sequence of SEQ ID NO: 69.

16. The method of claim 14, wherein the Apo-C3 modulating compound comprises:
a gap segment consisting of ten linked deoxynucleosides;
a 5′ wing segment consisting of five linked nucleosides; and
a 3′ wing segment consisting of five linked nucleosides,
wherein the gap segment is positioned between the 5′ wing segment and the 3′ wing segment, wherein each nucleoside of each wing segment comprises a 2′-O-methoxyethyl sugar, wherein each internucleoside linkage of said modified oligonucleotide is a phosphorothioate linkage, and wherein each cytosine residue of said modified oligonucleotide is a 5-methylcytosine.

17. A method of reducing triglycerides in a subject on Apo-C3 modulating therapy, the method comprising administering to the subject about 1 to about 4 capsules per day, each capsule comprising about 1 g of ethyl eicosapentaenoate.

18. The method of claim 17, wherein the subject has a fasting baseline triglyceride level of about 200 mg/dl to 499 mg/dl.

19. The method of claim 17, wherein the subject has a fasting baseline triglyceride level of at least 500 mg/dl.

20. The method of claim 17, wherein triglycerides are reduced in the subject with no increase in an LDL-C level in the subject.

21. The method of claim 20, wherein the reduction in triglycerides and the no increase in LDL-C level is in comparison to baseline or to a second subject or subject group that has received Apo-C3 modulating therapy but not the ethyl eicosapentaenoate.

22. The method of claim 17, wherein ethyl eicosapentaenoate represents at least about 90%, by weight of all fatty acids (and/or derivatives thereof) present.

23. The method of claim 22, wherein ethyl eicosapentaenoate represents at least about 95%, by weight of all fatty acids (and/or derivatives thereof) present.

24. The method of claim 23, wherein ethyl eicosapentaenoate represents at least about 96%, by weight of all fatty acids (and/or derivatives thereof) present.

25. The method of claim 17, wherein docosahexaenoic acid and its esters represent no more than about 20%, by weight of all fatty acids (and/or derivatives thereof) present in the capsule.

26. The method of claim 25, wherein docosahexaenoic acid and its esters represent no more than about 10%, by weight of all fatty acids (and/or derivatives thereof) present in the capsule.

27. The method of claim 26, wherein docosahexaenoic acid and its esters represent no more than about 5%, by weight of all fatty acids (and/or derivatives thereof) present in the capsule.

28. The method of claim 27, wherein docosahexaenoic acid and its esters represent no more than about 3%, by weight of all fatty acids (and/or derivatives thereof) present in the capsule.

29. The method of claim 17, wherein the Apo-C3 modulating therapy comprises administering to the subject an Apo-C3 modulating compound comprising the nucleobase sequence of any one of SEQ ID NOs: 1-337.

30. The method of claim 29, wherein the Apo-C3 modulating compound comprises the nucleobase sequence of SEQ ID NO: 69.

31. The method of claim 29, wherein the Apo-C3 modulating compound comprises:
a gap segment consisting of ten linked deoxynucleosides;
a 5′ wing segment consisting of five linked nucleosides; and
a 3′ wing segment consisting of five linked nucleosides,
wherein the gap segment is positioned between the 5′ wing segment and the 3′ wing segment, wherein each nucleoside of each wing segment comprises a 2′-O-methoxyethyl sugar, wherein each internucleoside linkage of said modified oligonucleotide is a phosphorothioate linkage, and wherein each cytosine residue of said modified oligonucleotide is a 5-methylcytosine.
